(19) United States
(12) Patent Application Publication
(10) Pub. No.: US 2025/0032692 A1
(43) Pub. Date: Jan. 30, 2025

(54) ISOLATION AND TREATMENT OF AN ORGAN OR TUMOR

(71) Applicant: DiNAQOR USA, Laguna Hills, CA (US)

(72) Inventors: Mark Dehdashtian, Irvine, CA (US); Christopher Aguayo, Irvine, CA (US); Johannes Holzmeister, Zurich (CH); Ole Blank, Zurich (CH); Maximilian Emmert, Zurich (CH); Nikola Cesarovic, Zurich (CH)

(21) Appl. No.: 18/918,476

(22) Filed: Oct. 17, 2024

Related U.S. Application Data
(63) Continuation-in-part of application No. 18/665,833, filed on May 16, 2024.
(60) Provisional application No. 63/502,635, filed on May 16, 2023.
(60) Provisional application No. 63/580,366, filed on Sep. 2, 2023.

(51) Int. Cl. A61M 1/36; A61M 25/10

(52) U.S. Cl. CPC A61M 1/3615; A61M 2025/1095

(57) ABSTRACT

Systems and methods for perfusing a treatment solution into the blood stream of particular organs while isolating those organs from the systemic vasculature as a whole. By deploying both collection and supply catheters, some having balloons on their distal ends, to particular locations within the body, various organs can be isolated so that the treatment solution can be delivered directly thereto. By isolating the organs, any side effects that may occur from a systemic introduction of the treatment solution are avoided. The method may be used to isolate not just organs, but specific tumors within certain organs for oncology treatment.

### RELATED APPLICATIONS

The present application is a continuation-in-part of U.S. patent application Ser. No. 18/665,833, filed May 16, 2024, which claims the benefit of priority to U.S. Provisional Ser. No. 63/502,635, filed May 16, 2023, and U.S. Provisional Ser. No. 63/580,366, filed Sep. 2, 2023, the contents of which are expressly incorporated herein.

### FIELD OF THE INVENTION

The present invention relates generally to treatment of certain diseases of the organs, and, in particular, to localized delivery of therapeutic agents to various organs and systems for such delivery.

### BACKGROUND OF THE INVENTION

Despite pharmacologic advances in the treatment of various organ conditions, such as liver cirrhosis or lung incapacity, challenges remain unacceptably high. Furthermore, certain therapeutic approaches are not suitable for many patients. Alternative approaches, such as gene therapy and cell therapy, have attracted increased attention due to their potential to be uniquely tailored and efficacious in addressing the root cause pathogenesis of many diseases.

Nevertheless, issues related to delivery, including vector efficiency, dose, specificity, and safety remain. For instance, systemic introduction of chemical or biological treatments often carries unintended consequences. As such, there is a need for further research directed to ways of achieving a more targeted, homogenous delivery of drugs suitable for treatment of various conditions that are also effective, well tolerated, and minimally invasive.

### SUMMARY OF THE INVENTION

The present application describes a so-called Loco-Regional Perfusion System and methods of perfusing one or more drugs in an isolated organ or tumor during beating heart of a patient. In some embodiments, the method comprises positioning at least one drug delivery catheter in an artery leading to the organ or tumor(s).

A disclosed system for perfusing a treatment solution to part of or all of an organ or tumor within the organ of a human body comprises at least one balloon collection catheter having a length sufficient to extend from outside the body into a patient's vasculature such that a distal end of the collection catheter is located at a return location in a venous vessel leading from the organ or tumor. The return location is positioned so as to collect substantially all returned blood from the organ or area of the tumor. A balloon on the distal end of the collection catheter is inflatable and sized to occlude the venous vessel, and a distal tip of the collection catheter has apertures for receiving venous blood therethrough from the venous vessel into a lumen of the collection catheter. At least one supply catheter has a length sufficient to extend from outside the body into the patient's vasculature such that a distal end of the supply catheter is located at a supply location in an arterial vessel leading to the organ or tumor. The supply location is positioned so as to supply substantially all delivered blood to the organ or area of the tumor, and a distal tip of the supply catheter having an aperture for perfusing fluid from a lumen of the supply catheter therethrough into the arterial vessel. The system has an extracorporeal oxygenator fluidly connected to a proximal end of the collection catheter, the oxygenator enabling oxygenation of the venous blood and having a conduit fluidly connected on a second end to a pump such as a peristaltic or centrifugal pump. Also, a source of treatment solution is fluidly connected to the conduit between the oxygenator and the pump. Further, an output conduit fluidly connected from the pump to the lumen of the supply catheter, wherein an isolated circuit is created from the organ or tumor through the collection catheter, from there through the oxygenator and pump, and then through the supply catheter back to the organ or tumor, whereby blood can be removed from just the organ or tumor and returned just to the organ or tumor oxygenated and perfused with the treatment solution.

The organ may be the brain and the organ or tumor within the organ is a brain tumor, wherein the source of treatment solution includes a controlled supply of oncology drug at low pressure and flow. The organ may be a lung, and the treatment solution is a cancer treatment selected from the group consisting of an alkylating agent, a taxane, an albumin-bound paclitaxel, an antimetabolite, a vinca alkaloid, and a topoisomerase II inhibitor.

There may be multiple balloon collection catheters each have a length sufficient to deploy in different venous vessels leading from the organ or tumor. In an embodiment, the supply catheter is a balloon supply catheter having a balloon vessel, the balloon being sized to occlude the arterial vessel. Further, there may be multiple balloon supply catheters each have a length sufficient to deploy in different venous vessels leading to the organ or tumor as well as multiple balloon collection catheters each have a length sufficient to deploy in different venous vessels leading from the organ or tumor.

A disclosed method comprises positioning at least one drug collection catheter in a vein leading from the organ and or tumor in an organ and at least one drug supply catheter in an artery leading to the organ or tumor. The drug delivery catheter(s) and the drug collection catheter(s) together with the arteries of the organ, the venous system of the organ, and a membrane oxygenation device form a closed circuit. The method further comprises perfusing the drug(s) through the closed circuit, which isolates the circulation of the organ from the systemic circulation of the patient.

An exemplary method for perfusing a treatment solution to an organ or tumor within the organ of a human body comprises:
a. introducing at least one balloon collection catheter from outside the body into a patient's vasculature and advancing a distal end of the collection catheter to a return location in a venous vessel leading from the organ or tumor, the return location being located so as to collect substantially all returned blood from the organ or area of the tumor, a distal tip of the collection catheter having apertures for receiving venous blood therethrough from the venous vessel into a lumen of the collection catheter;
b. inflating a balloon on the distal end of the collection catheter within the venous vessel;
c. introducing at least one balloon supply catheter from outside the body into the patient's vasculature and advancing a distal end of the supply catheter to a supply location in an arterial vessel leading to the organ or tumor, the supply location being located so as to supply substantially all delivered blood to the organ or area of the tumor, a distal tip of the supply catheter having apertures for perfusing fluid from a lumen of the supply catheter therethrough into the arterial vessel;
d. inflating a balloon on the distal end of the supply catheter within the arterial vessel;
c. connecting a proximal end of the collection catheter to a first end of an extracorporeal oxygenator, the oxygenator enabling oxygenation of the venous blood and having a conduit connected on a second end to a pump such as a peristaltic or centrifugal pump;
f. connecting a source of treatment solution to the conduit between the oxygenator and the pump; and
g. connecting an output conduit from the pump to the lumen of the supply catheter, wherein an isolated circuit is created from the organ or tumor through the collection catheter, from there through the oxygenator and pump, and then through the supply catheter back to the organ or tumor, whereby blood can be removed from just the organ or tumor and returned just to the organ or tumor oxygenated and perfused with the treatment solution.

The method may further comprise applying negative pressure at the drug collection catheter. In some embodiments, the negative pressure ranges from about-100 mm Hg to 0 mm Hg. Alternatively, the pressure may be increased at the drug supply catheter by about 100 mm Hg to 160 mm Hg.

One or more of the drug delivery catheter(s) or the drug collection catheter(s) are introduced percutaneously. For instance, the drug delivery catheter(s) is/are positioned via antegrade intubation. In some embodiments, the collection catheter(s) is/are positioned in the organ vein via the inferior vena cava of the patient. Typically, the supply line is introduced in a retrograde fashion (with the end of the catheter pointed opposite the direction of blood flow to the heart, and femoral access), while the return line is antegrade (with the end of the catheter pointed toward the direction of blood flow to the heart, and jugular access). However, the access sites may also change as well as the direction of access.

The membrane oxygenation device is preferably positioned between the collection catheter(s) and the drug delivery catheter(s). One or more of the drug delivery catheter(s) or the drug collection catheter(s) are sealed by a balloon to reduce or prevent leakage to systemic circulation.

The method may further comprise circulating blood through the closed circuit. In some embodiments, the blood comprises autologous blood, matched blood from donors, or a combination thereof. Blood components such as serum or plasma are chosen according to one or more parameters, such as presence or absence of selected antibodies. In some embodiments, about 1000 mL, about 800 mL, about 600 mL, about 400 mL, about 200 mL, about 100 mL, or about 50 mL of blood is circulated through the closed circuit.

Reduction of the blood temperature 3-4 degree may be included during the secondary closed circulation. An oxygenator through which the blood circulates may have a heater that controls the blood temperature going back into the body.

The perfusing may occur over a duration of about 5 minutes to about 5 hours, about 15 minutes to about 4 hours, about 30 minutes to about 3 hours, or about 1 hour to about 2 hours. For instance, the perfusing occurs for at least 60 minutes. The duration is a function of the drug concentration and effectiveness. The perfusing may occur at a flow rate of about 75 mL/min to about 750 mL/min, about 150 mL/min to about 500 mL/min, or about 200 ml/min to about 300 mL/min.

### Oncology Applications:

The circulation isolation systems described herein can isolation various organs or tumors in the human body by creating and controlling a secondary circulation of a cancer treatment medication for a duration of a procedure not to exceed 5 hours (0-5 hours), with control over the appropriate parameters such as:
Concentration,
Temperature,
Time,
Flow rate.

Exemplary cancer treatments include isolating a tumor and treating just the tumor with a chemotherapy drug. This helps avoid negative complications common in more systemic applications of such powerful drugs.

For the Cranial Cavity and other cavities of the body, the isolation systems could further target and or isolate just a tumor or tumors with perfusion of higher concentration of the therapeutic agent for more effectiveness.

### Alzheimer Applications:

The isolation systems can also target specific parts of the cranial cavity related to Alzheimer by delivery of the agent to specific and targeted area for more effectiveness. For instance, cholinesterase inhibitors such as galantamine, rivastigmine, and donepezil are prescribed for mild to moderate Alzheimer's symptoms. These drugs may help reduce or control some cognitive and behavioral symptoms.

The drug may also comprise a therapeutic polynucleotide sequence, and the therapeutic polynucleotide sequence may be present in one or more viral vectors. In some embodiments, the one or more viral vectors is selected from the group consisting of an adeno-associated virus, an adenovirus, a retrovirus, a herpes simplex virus, a bovine papilloma virus, a lentiviral vector, a vaccinia virus, a polyoma virus, a sendai virus, orthomyxovirus, paramyxovirus, papovavirus, picornavirus, pox virus, alphavirus, variations thereof, and combinations thereof.

The viral vector may be an adeno-associated virus (AAV). In some embodiments, the AAV is one or more of AAV1, AAV2, AAV3, AAV4, AAV5, AAV6, AAV7, AAV8, AAV9, AAV10, AAV11, AAV12, variations thereof, and combinations thereof.

The therapeutic polynucleotide sequence may comprise a nucleic acid sequence encoding to a protein, antisense RNA, ncRNA, or miRNA for treatment of an organ condition. In some embodiments, the protein corresponds to a gene expressed in a human heart. In some embodiments, the protein is one or more of SERCA2, MyBPC3, MYH7, PKP2, dystrophin, FKRP, or a combination or variation thereof. In some embodiments, the therapeutic polynucleotide sequence comprises a promoter.

The present application discloses methods of maintaining perfusion of a perfusate through a closed circuit in an organ of a patient while the heart beats during the perfusion. The method comprises positioning at least one drug delivery catheter in an artery leading to the organ. The method further comprises positioning a drug collection catheter in a vein leading from the organ. The drug delivery catheter(s) and the drug collection catheter together with the arteries of the organ, the venous system of the organ, and a membrane oxygenation device form a closed circuit through the organ that is isolated from the patient's systemic circulation. The membrane oxygenation device may fluidly couple to the drug delivery catheter(s). The closed circuit may also have an oxygen source and a pump configured to drive fluid flow through the drug delivery catheter(s). The method may comprise flowing perfusate through the closed circuit by introducing the perfusate into the organ via the drug delivery catheter(s) and collecting the perfusate via the collection catheter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Features and advantages of the present invention will become appreciated as the same become better understood with reference to the specification, claims, and appended drawings wherein:

FIG. 1 is a schematic view of the organs of the chest as well as several of the larger blood vessels;

FIG. 2 is a schematic view of the upper chest showing the heart in section and the respective chambers therein, as well as the main vascular pathways to and from the lungs;

FIG. 3 is a schematic view of one vascular pathway for introducing treatment solutions to the body;

FIG. 4 is a schematic view of an extracorporeal perfusion circuit (EPC) for introducing treatment solutions to the body through the femoral vein;

FIG. 5 is a schematic view similar to FIG. 2 showing catheter pathways for introducing treatment solutions to one of the lungs;

FIG. 6 is an anterior view of the lungs showing a typical division of both left and right lungs into lobes separated by fissures indicated thereon;

FIG. 6A is an anterior view of the lungs showing a typical division of the lobes into smaller segments superimposed thereon;

FIG. 7 is an anterior view of the lungs and heart along with the vasculature therebetween and into the lungs;

FIG. 8 is an enlargement of a segment of the left lung and heart along with the main vessels, showing placement of exemplary supply and return catheters for isolating one segment of the left lung;

FIG. 9 is an anterior view of the left lung from the outside after a test treatment of the isolated segment with contrast fluid;

FIG. 10A is a schematic view of the liver and hepatic veins for collecting blood therefrom to the heart, FIG. 10B shows the portal veins for introducing De-Oxygenated blood from digestive system to the liver, and FIG. 10C shows the hepatic arteries that deliver red blood to the liver;

FIG. 11A is a schematic view of the pancreas and primary veins for collecting blood therefrom to the heart along with locations for positioning collection catheter balloons, and FIG. 11B shows the primary arteries for introducing blood to the pancreas along with locations for positioning supply catheter balloons;

FIG. 12A is a schematic view of the spleen and primary veins for collecting blood therefrom to the heart along with locations for positioning collection catheter balloons, and FIG. 12B shows the primary arteries for introducing blood to the spleen along with locations for positioning supply catheter balloons;

FIG. 13A is a schematic view of the head and primary veins for collecting blood therefrom to the heart along with locations for positioning collection catheter balloons, and FIG. 13B shows the primary arteries for introducing blood to the head along with locations for positioning supply catheter balloons;

FIG. 14A is a schematic view of one of the eyes and primary veins for collecting blood therefrom to the heart along with locations for positioning collection catheter balloons, and FIG. 14B shows the primary arteries for introducing blood to the eye along with locations for positioning supply catheter balloons;

FIG. 15A is a schematic view of the breasts and primary veins for collecting blood therefrom to the heart along with locations for positioning collection catheter balloons, and FIG. 15B shows the primary arteries for introducing blood to the breasts along with locations for positioning supply catheter balloons;

FIG. 16A is a schematic view of the right breast and primary veins for collecting blood therefrom to the heart along with locations for positioning collection catheter balloons, and FIG. 16B shows the primary arteries for introducing blood to the right breast along with locations for positioning supply catheter balloons;

FIG. 17 is a schematic view of the female uterus, bladder and reproductive system along with an overview of the blood vessels therein;

FIG. 18 is a diagram of the blood flow to the female ovaries along with locations for positioning both collection and supply catheter balloons, and FIG. 18A is a detail thereof;

FIG. 19A is a schematic view of the female uterus and primary veins for collecting blood therefrom to the heart along with locations for positioning collection catheter balloons, and FIG. 19B shows the primary arteries for introducing blood to the female uterus along with locations for positioning supply catheter balloons;

FIG. 20A is a schematic view of the female bladder and primary veins for collecting blood therefrom to the heart along with locations for positioning collection catheter balloons, and FIG. 20B shows the primary arteries for introducing blood to the female bladder along with locations for positioning supply catheter balloons;

FIG. 21A is a diagram of the large veins located in the male crotch region, and FIG. 21B is a diagram of the large arteries therein;

FIG. 22A is a schematic view of the male bladder and primary veins for collecting blood therefrom to the heart along with locations for positioning collection catheter balloons, and FIG. 22B shows the primary arteries for introducing blood to the male bladder along with locations for positioning supply catheter balloons;

FIG. 23A is a schematic view of the male prostate and primary veins for collecting blood therefrom to the heart along with locations for positioning collection catheter balloons, and FIG. 23B shows the primary arteries for introducing blood to the male prostate along with locations for positioning supply catheter balloons;

FIG. 24 is a diagram of the blood flow to the male testicles along with locations for positioning both collection and supply catheter balloons;

FIG. 25 is a schematic view of the upper thorax, neck and brain along with an overview of the blood vessels therein, along with a typical position of a brain tumor;

FIG. 25A is a schematic view of the brain and primary veins for collecting and introducing blood thereto along with locations for positioning collection and supply catheter balloons to isolate and treat the tumor;

FIG. 25B is a schematic view of the brain and primary veins for collecting and introducing blood thereto along with alternative locations for positioning collection and supply catheter balloons to isolate and treat the tumor;

FIG. 26A is a schematic view of the colon with labels for different sections thereof, and FIG. 26B shows an overview of the blood vessels therein;

FIG. 27A is a schematic view of the colon and primary veins for collecting blood therefrom to the heart, and FIG. 27B shows the primary veins isolated along with locations for positioning collection catheter balloons;

FIG. 28A is a schematic view of the colon and primary arteries for introducing blood from the heart, and FIG. 28B shows the primary arteries isolated along with locations for positioning supply catheter balloons;

FIG. 29 is another schematic view of the colon with labels for different sections thereof and the colorectal region outlined;

FIG. 29A is a schematic view of the primary veins in the colorectal region along with locations for positioning collection catheter balloons to isolate and treat diseases therein;

FIG. 29B is a schematic view of the primary arteries in the colorectal region along with locations for positioning supply catheter balloons to isolate and treat diseases therein;

FIG. 30A is a schematic view of the primary veins in the stomach region along with locations for positioning collection catheter balloons to isolate and treat diseases therein;

FIG. 30B is a schematic view of the primary arteries in the stomach region along with locations for positioning supply catheter balloons to isolate and treat diseases therein;

FIG. 31 is another schematic view of the esophagus with labels for different sections thereof and an overview of nearby blood vessels;

FIG. 31A is a schematic view of the primary veins in the esophagus along with locations for positioning collection catheter balloons to isolate and treat diseases therein;

FIG. 31B is a schematic view of the primary arteries in the esophagus along with locations for positioning supply catheter balloons to isolate and treat diseases therein;

FIG. 32 is another schematic view of the thyroid gland and an overview of nearby blood vessels;

FIG. 32A is a schematic view of the primary veins near the thyroid gland along with locations for positioning collection catheter balloons to isolate and treat diseases therein;

FIG. 32B is a schematic view of the primary arteries near the thyroid gland along with locations for positioning supply catheter balloons to isolate and treat diseases therein;

FIG. 33 is a broken elevational view of an exemplary collection catheter of the present application;

FIG. 33A is an enlargement of a distal balloon on the collection catheter of FIG. 33, and FIG. 33B shows the balloon in an extended state;

FIGS. 34A-34C illustrate a sequence of deployment of the distal balloon of the collection catheter of FIG. 33 and withdrawal of fluid distal to the catheter;

FIG. 35 is a broken elevational view of an exemplary supply catheter of the present application;

FIG. 35A is an enlargement of a distal balloon on the supply catheter of FIG. 35, and FIG. 35B shows the balloon in an extended state; and

FIGS. 36A-36C illustrate a sequence of deployment of the distal balloon of the supply catheter of FIG. 35 and perfusion of fluid distal to the catheter.

### DETAILED DESCRIPTION OF THE PREFERRED EMBODIMENTS

The present invention provides a system for perfusing a treatment solution into the blood stream of particular organs while isolating those organs from the systemic vasculature as a whole. Both collection and supply catheters having balloons on their distal ends to particular locations within the body, various organs can be isolated so that the treatment solution can be delivered directly thereto. By isolating the organs, any side effects that may occur from a systemic introduction of the treatment solution are avoided.

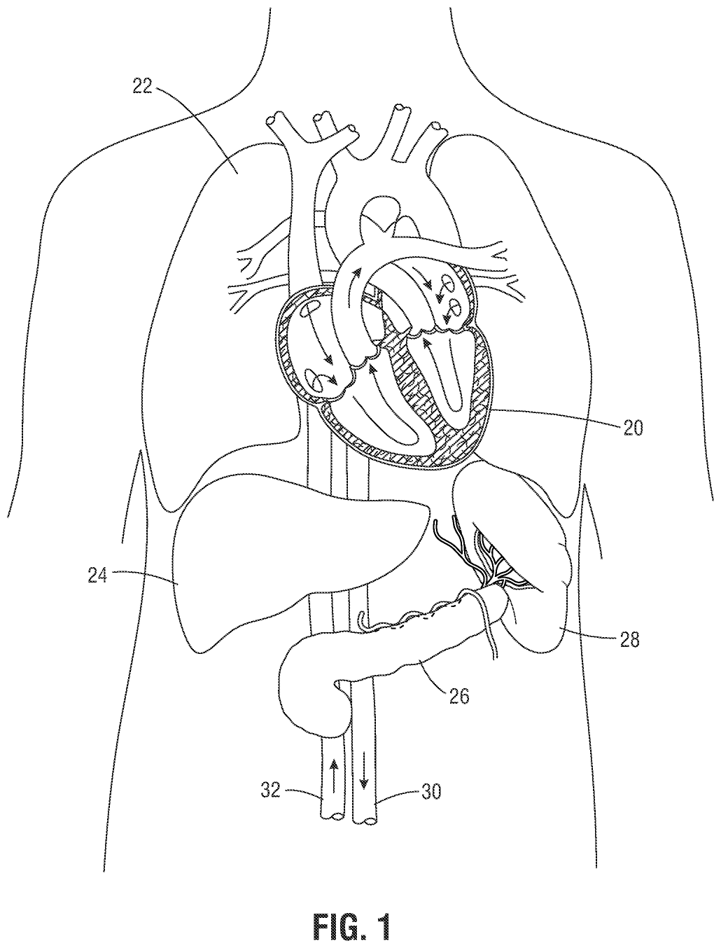

FIG. 1 is a schematic view of the organs of the chest as well as several of the larger blood vessels. As is well known, the heart 20 comprises four chambers for receiving venous blood and directing the venous blood to the lungs, and then receiving oxygenated blood from the lungs and directing that blood to the body's vasculature. The general flow of love is shown. The lungs 22 are located just behind the heart, the liver 24 below the lungs, and the pancreas 26 and spleen 28 farther down the abdomen. The heart 20 pumps blood downward through the abdominal aorta 30, and majority of the venous blood from the lower extremities passes through the vena cava 32.

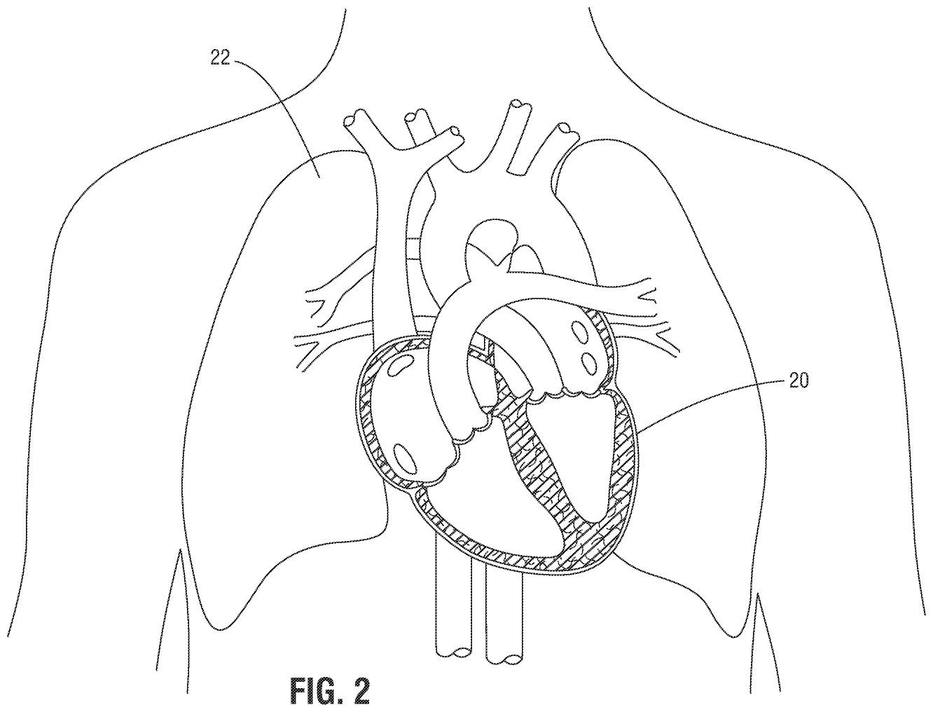

FIG. 2 is a schematic view of the upper chest showing the heart 20 in section and the respective chambers therein, as well as the main vascular pathways to and from the lungs 22.

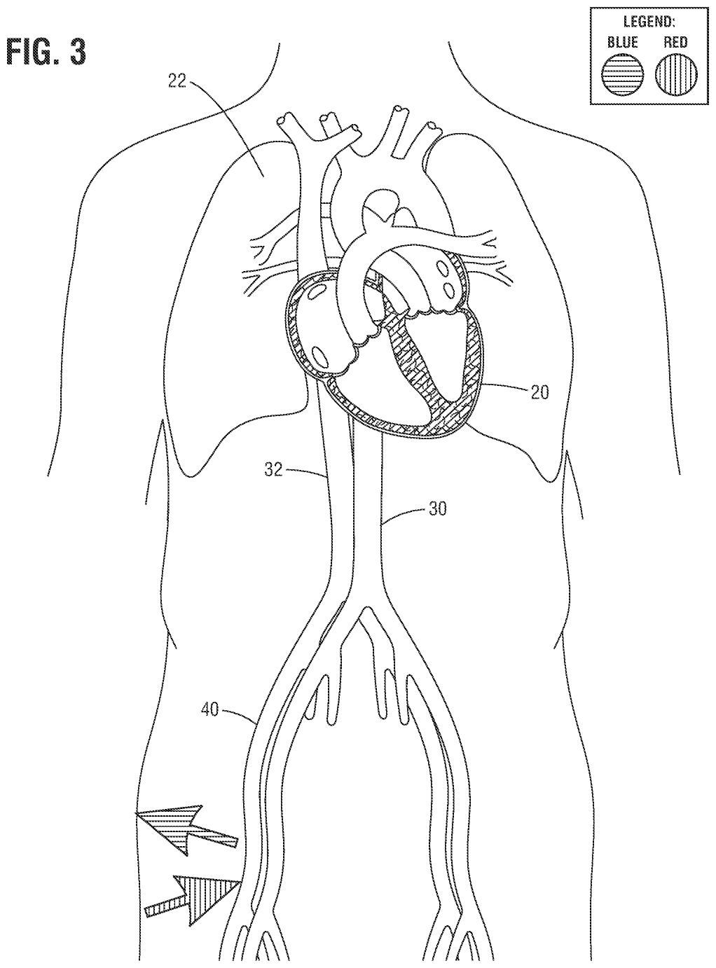

FIG. 3 is a schematic view of one vascular pathway for introducing treatment solutions to the body. Namely, a blood collection pathway indicated by a large blue arrow exits the body after the femoral vein 40. Likewise, a blood supply pathway indicated by a large red arrow is introduced into the body at the femoral vein 40 as well.

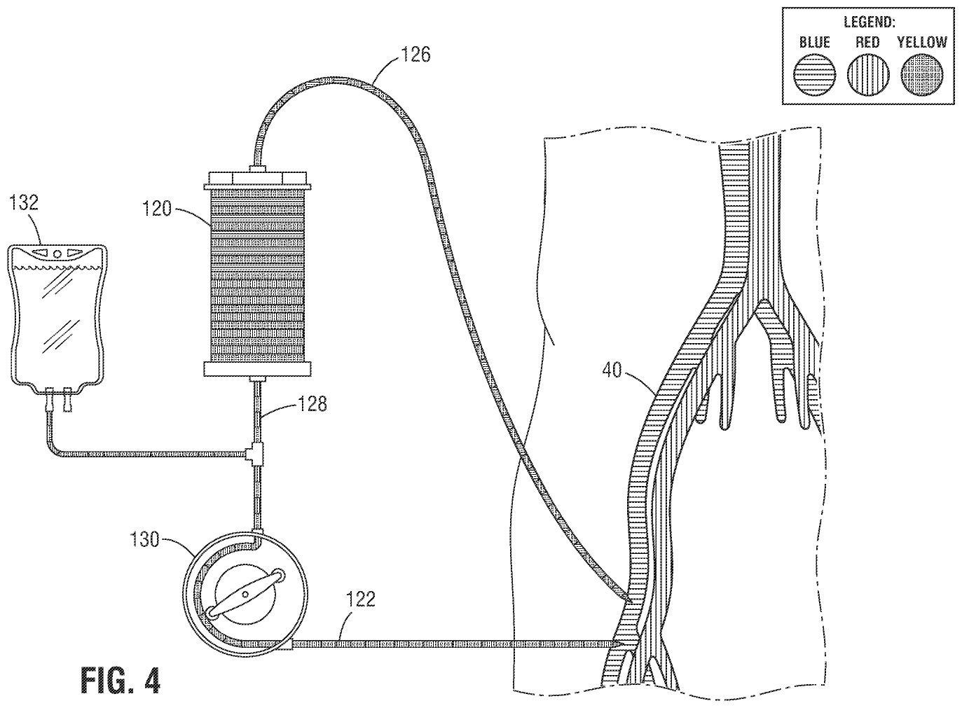

FIG. 4 is a schematic view of an extracorporeal perfusion circuit EPC for introducing his treatment solutions to the body through the femoral vein 40.

The extracorporeal perfusion circuit EPC includes a membrane oxygenation device 120, a blood gas analysis (BGA) monitor (not shown), and a pressure monitor (not shown). The extracorporeal perfusion circuit EPC may be assembled by positioning a first supply catheter 122 in the arterial system adjacent the target organ, and positioning a collection catheter 126 in the venous system adjacent the target organ. (In this example, both catheters are shown entering the femoral vein, from where the supply catheter may then be subsequently redirected through a heart chamber into the arterial system.) The first catheter 122 and the collection catheter 126, together with the arteries, venous system, the membrane oxygenation device 120, and one or more optional additional components form a closed circuit. This closed circuit may isolate or substantially isolate the organ circulation of the patient from the systemic circulation of the patient.

A proximal end of the collection catheter 126 connects to a first end of the extracorporeal oxygenator 120 which, in turn, has a conduit 128 connected on a second end to a peristaltic pump 130. A source of treatment solution 132 connects to the conduit 128 between the oxygenator 120 and the peristaltic pump 130. As an alternative, a centrifugal pump (not shown) may also be used.

The first catheter 122 and the collection catheter 126 may be introduced percutaneously and in a minimally invasive manner. In some embodiments, the first catheter 122 may be introduced the femoral vein 40, but also via the subclavian vein (not shown). The first catheter 122 may be referred to herein as a “drug delivery catheter” and the collection catheter 126 may be referred to herein as a “drug collection catheter” when the catheters are used for drug delivery.

The first catheter 122 and the collection catheter 126 may each be a standard perfusion catheter that may optionally include a standard guidewire and perfusion pump. Each catheter is capable of delivering a perfusate to the organ, which may contain, for example, a drug to be delivered to the organ.

The extracorporeal perfusion circuit EPC may further comprise one or more additional components, such as, without limitations, one or more pumps, one or more suction mechanisms, one or more perfusates, and combinations thereof. For example, the extracorporeal perfusion circuit EPC is depicted as including a membrane oxygenation device 120. A pressure monitor may be used to control the perfusion rate (i.e., flowrate) and ensure safety by continuously monitoring the arterial pressure. A first pressure sensor may be co-inserted with the first catheter 122 to measure the pressure at the catheter tip. The extracorporeal perfusion circuit EPC may further include a BGA monitor that is operatively coupled to the membrane oxygenation device 120 to measure, for example, the gas concentrations in the perfusate (e.g., when the perfusate contains blood) prior to perfusion via the first catheter 122 and/or after the perfusate is collected by the collection catheter 126. The membrane oxygenation device 120 and one or more additional components may be placed between the collection catheter 126 and the first catheter 122.

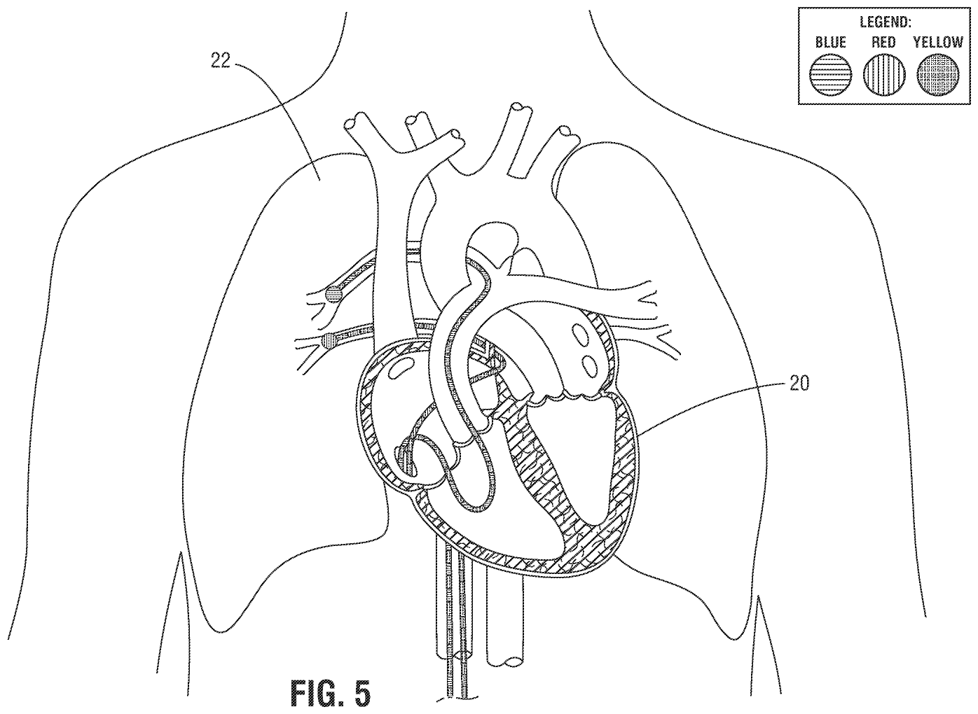

FIG. 5 is a schematic view similar to FIG. 2 showing catheter pathways for introducing treatment solutions to one of the lungs. The red collection line occludes and receives blood and treatment solution if the circuit has been running from the right lung via the right pulmonary vein—the return location. The catheter will pass across the septum between the right atrium and the right pulmonary vein. After circulating outside the body and being treated (and potentially de-oxygenated), the blood and payload (treatment solution) enters through the femoral vein as explained and is supplied to the pulmonary artery—the supply location—by the tricuspid valve and pulmonic valves. The return location collects substantially all returned blood from the right lung, and the supply location supplies substantially all delivered blood to the right lung.

It should be noted that in the lungs, normally the “venous” blood is that returned from the lungs, which is oxygenated by the alveoli and capillaries in the lungs. Consequently, the terms venous and arterial are a broader set of terms for blood from and to an organ, even the lungs. For all other organs, the arterial blood is oxygenated and the venous blood de-oxygenated. For the lungs, the opposite is true. So, in the lungs a venous vessel carries oxygenated blood from the lungs back into the systemic circulation, while an arterial vessel carries de-oxygenated blood to the lungs. When isolated by the systems described herein, a lung, lobe or segment of a lung receives blood and treatment solution from the supply catheter and the return catheter collects oxygenated blood and any remaining treatment solution.

In contrast to isolation and treatment of all other organs, the oxygenated or red blood collected from the lungs is circulated outside the body and either not oxygenated or de-oxygenated along with the addition of treatment solution. It is not believed necessary or even safe to further oxygenate red blood, and so there is typically no oxygenator in the circuit. Devices for de-oxygenating blood are known, such as disclosed in U.S. Pat. No. 8,569,052 developed at the U. of Pittsburgh. These devices typically route blood through porous tubes within an oxygen scavenger. Such a device may be utilized in an isolation circuit for treating the lungs, as described herein, wherein the de-oxygenated blood is then circulated back to be re-oxygenated by the lungs.

### Isolation and Treatment of the Lungs

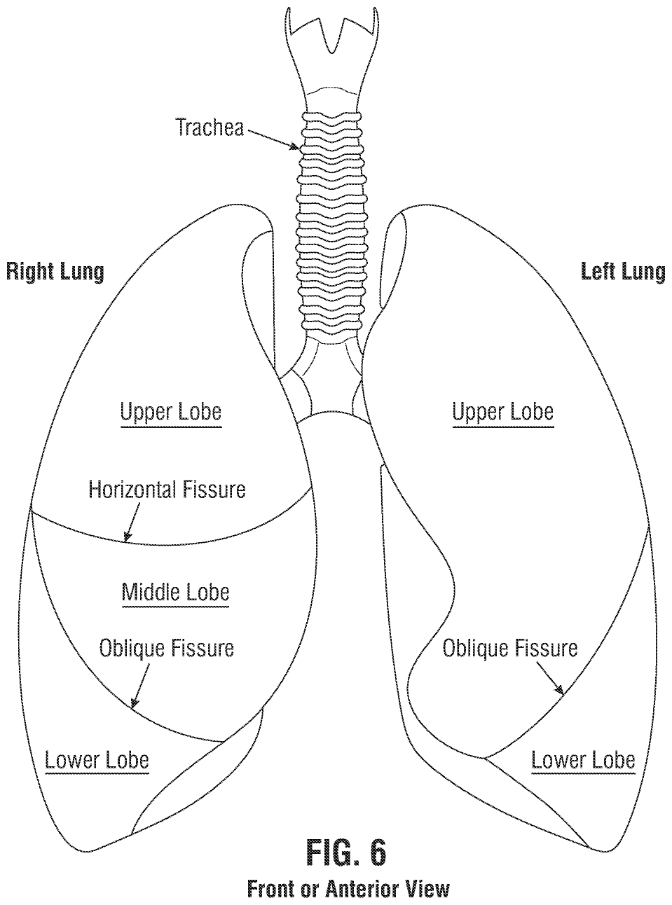

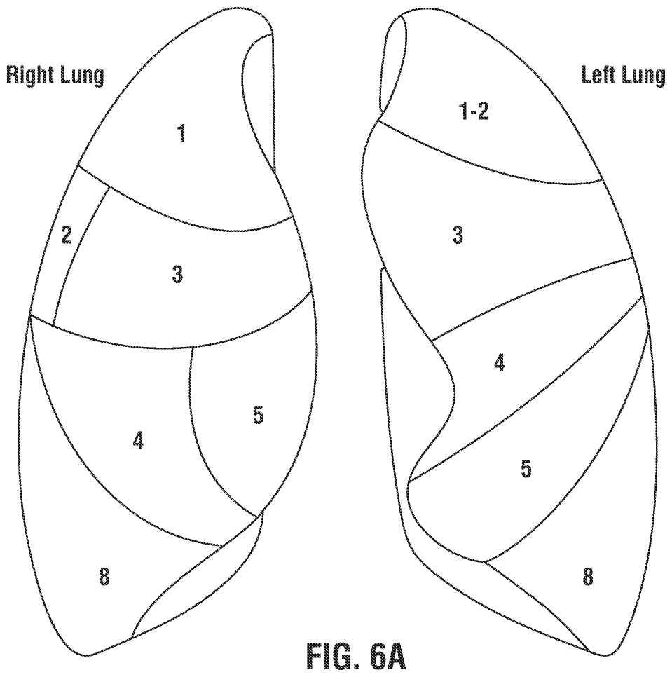

FIG. 6 is an anterior view of the lungs showing a typical division of both left and right lungs into lobes separated by fissures indicated thereon. With a focus on Oncology therapy, the lungs may be segregated into various lobes and then smaller segments. The present techniques contemplate isolating 5 Lobes: In the Left Lung, the Upper Lobe, and Lower Lobe, and for the Right Lung, Upper, Middle and Lower Lobes. So-called “fissures” separate the lobes and any one lobe may be isolated due to the separate blood supply and return vessels to each.

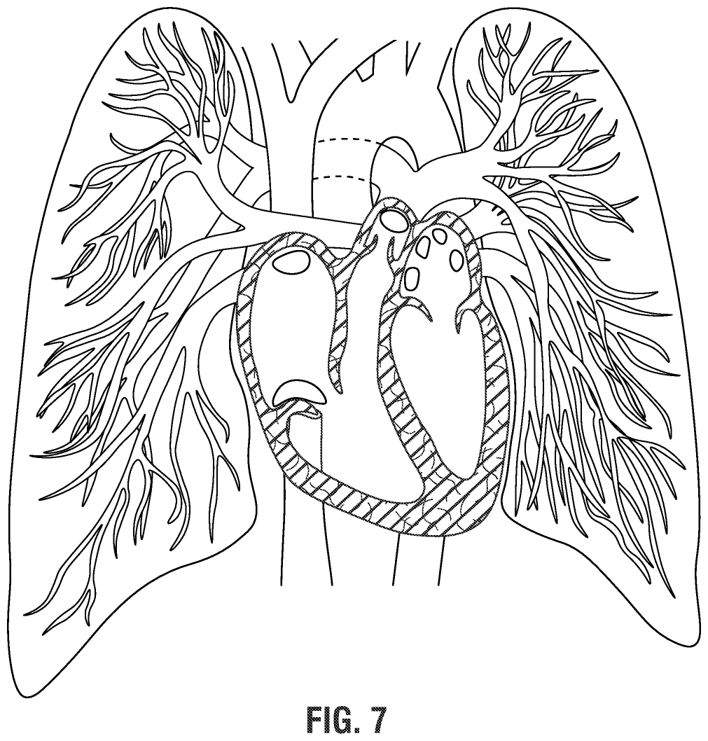

Moreover, the lobes may be further divided into smaller segments which also have unique blood supply and return vessels. FIG. 6A is an anterior view of the lungs showing a typical division of the lobes into smaller segments superimposed thereon, and FIG. 7 is an anterior view of the lungs and heart along with the vasculature therebetween and into the lungs.

The 5 Lobes have a total of 24 possible segments combined that theoretically can be isolated. Clinical investigation has so far identified the following 17 discrete segments that can be isolated to establish supply and return paths for isolating these segments by using the treatment systems described herein:
Left Lung Upper Lobe: Apical and Posterior (1-2), and Anterior (3).
Left Lung Lingula: Superior (4) and Inferior (5).
Left Lung Lower Lobe: Anterior Basal (8), Lateral Basal, Posterior Basal (others not visible in anterior view).
Right Lung Upper Lobe: Apical (1), Posterior (2) and Anterior (3).
Right Lung Middle Lobe: Lateral (4) Medial (5).
Right Lung Lower Lobe: Superior Basal, Medial Basal, Anterior Basal (8), Lateral Basal, Posterior Basal (others not visible in anterior view).

Due to the complex division of airway tubes branching off the trachea into the two lungs, dedicated blood vessel branches lead to particular small segments within the lungs. As such, careful placement of the supply and return catheters enables isolation of one or more specific segments to provide targeted oncology treatment,

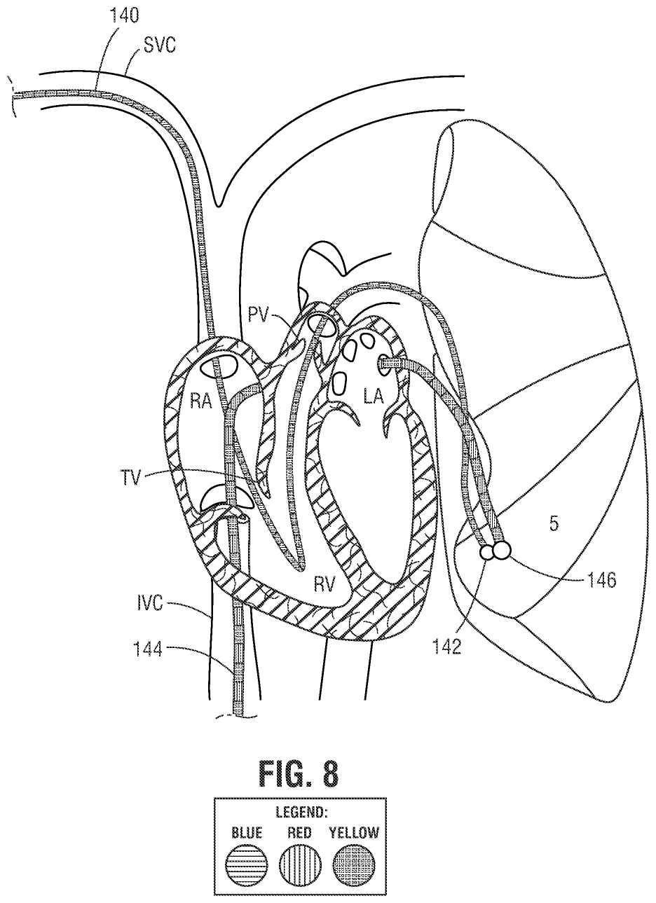

FIG. 8 is an enlargement of a segment of the left lung and heart along with the main vessels, showing placement of exemplary supply and return catheters for isolating one segment of the left lung; in this case the Inferior segment (5) within the Left Lung Lingula. A supply catheter 140 is shown passing through an access pathway that includes the Superior Vena Cava (SVC), through the Right Atrium (RA) and across the Tricuspid Valve (TV) into the Right Ventricle (RV), and from there across the Pulmonary Valve (PV) in the Pulmonary trunk to the pulmonary arteries that lead to the Inferior segment (5) within the Left Lung Lingula. This access pathway is different than that shown in FIG. 5, where both catheters pass up through the Inferior Vena Cava (IVC), but the two catheters both travel down the vasculature and are inserted into the body in the thigh region. The supply catheter 140 is shown carrying blue or de-oxygenated blood to distinguish it from the red blood collected from the lungs, though as mentioned above, the supply blood may be simply the un-oxygenated red blood collected from the lungs. One or more such supply catheters 140 may be utilized depending on the number of large branch pulmonary arteries leading to the respective segment. Each supply catheter 140 has an inflatable balloon 142 on a distal end thereof to anchor the catheter and block flow around the catheter. The distal end of the catheter 140 is located at a supply location such that substantially all blood mixed with the treatment therapy is thus delivered to the target segment.

One or more return catheters 144 also has/have a distal anchoring balloon 146 thereon and is positioned within the main return vessel from the target segment so that substantially all returned blood from the segment is captured. The return catheter 144 is shown passing through one of the main arterial vessels leading to the Left Atrium (LA), and from there through the Atrial Septal Wall (SW) to the Right Atrium (RA), and then down through the Inferior Vena Cava (IVC). The return catheter 144 thus collects all of the treatment therapy mixed with the blood that is applied to the target segment to prevent wider dispersion through the vasculature.

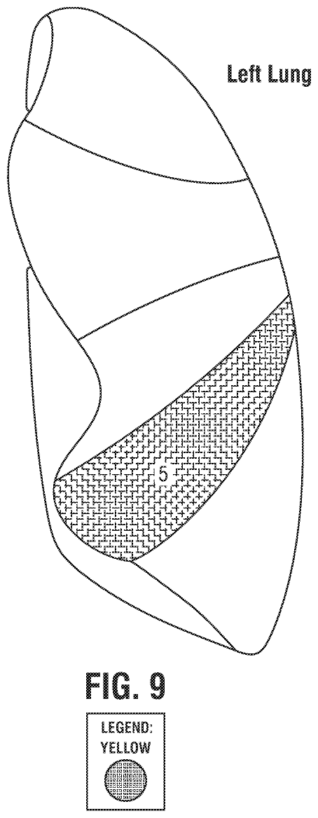

FIG. 9 is an anterior view of the left lung from the outside after a test treatment in an animal of the isolated Inferior segment (5) within the Left Lung Lingula with contrast fluid. The entire segment shows the contrast color without any being transferred to the adjacent segments, thus conforming the isolating aspect of the technique. This targeted application of the particular therapy demonstrates that the disclosed techniques can be used to treat a specific region in the lungs rather than the entire lung or broader system, which greatly reduces negative collateral effects.

Common treatments for lung cancer depend on the cancer's specific pathology, staging, and the patient's performance status (e.g., ability to breath). Traditional treatment options are surgery, chemotherapy, immunotherapy, radiation therapy, and palliative care. Intravascular techniques for localized delivery of chemotherapeutic agents have also been used to treat lung cancer, and include cancer therapy such as arterial chemoembolization, bronchial artery infusion (BAI), isolated lung perfusion (ILP), and lung suffusion.

Chemotherapeutics most often used for non-small cell lung cancer (NSCLC) include:

Alkylating agents such as Cisplatin or Carboplatin that contain the metal platinum,

Taxanes such as Paclitaxel (Taxol)

Albumin-bound paclitaxel (nab-paclitaxel, Abraxane) or Docetaxel (Taxotere),

Antimetabolites such as Gemcitabine (Gemzar) or Pemetrexed (Alimta), Vinca alkaloids such as Vinorelbine (Navelbine), and

Topoisomerase II inhibitor such as Etoposide (VP-16).

Other chemotherapeutics approved for the treatment of non-small cell lung cancer in the United States include methotrexate, paclitaxel albumin-stabilized nanoparticle formulation, afatinib dimaleate, everolimus, alectinib, pemetrexed di sodium, atezolizumab, bevacizumab, carboplatin, ceritinib, crizotinib, ramucirumab, docetaxel, erlotinib hydrochloride, gefitinib, afatinib dimaleate, gemcitabine hydrochloride, pembrolizumab, mechlorethamine hydrochloride, methotrexate, vinorelbine tartrate, necitumumab, nivolumab, paclitaxel, ramucirumab, and osimertinib, and the combinations carboplatin-taxol and gemcitabine-cisplatin (https://www.cancer.gov/aboutcancer). Drugs approved for the treatment of small cell lung cancer include methotrexate, everolimus, doxorubicin hydrochloride, etoposide phosphate, topotecan hydrochloride, mechlorethamine hydrochloride, and topotecan (https://www.cancer.gov/aboutcancer). Lung cancer such as small cell lung cancer can sometimes be treated with a combination of radiation therapy and one or more chemotherapeutics.

### Isolation and Treatment of the Liver

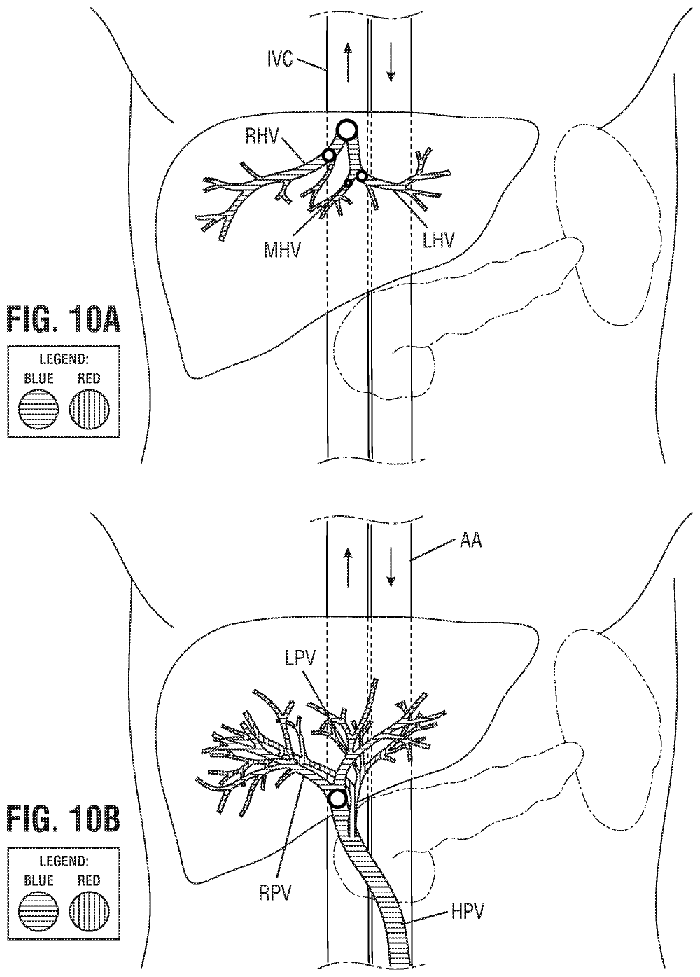

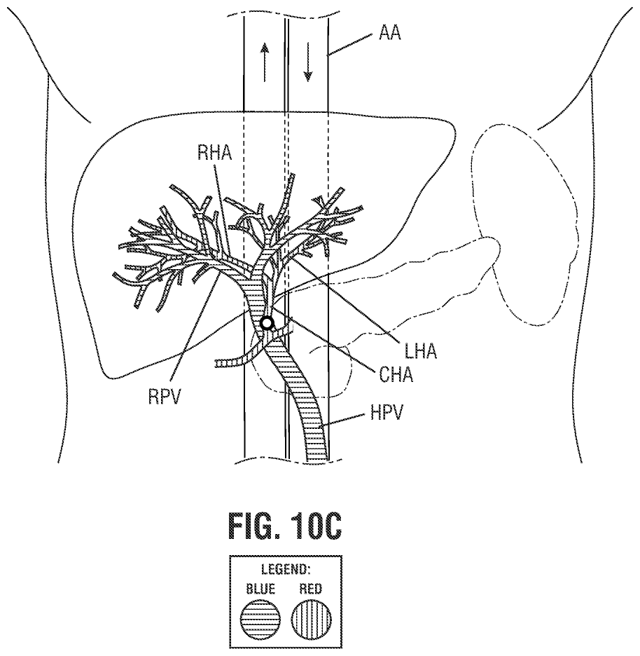

FIGS. 10A-10C are schematic views of the liver and primary veins for collecting blood therefrom to the heart. There are 2 blood supply vessels to Liver: 1) Oxygenated blood from Abdominal Aorta (AA) to Celiac Trunk to Hepatic Artery to Liver, and 2) De-Oxygenated blood from digestive system (Stomach-Large & small cologne-Pancreas-Spleen, etc.,) to Hepatic Portal Vein to Liver.

There are 3 options to isolate the Liver with the Loco-Regional Perfusion System described herein. First, Oxygenated blood with the treatment therapy may be administered from the Abdominal Aorta (AA) to the Celiac Trunk to Hepatic Artery to the Liver (Percutaneous approach). Occlusion and introduction of treated blood and payload may be done via the Common Hepatic Artery (CHA), as shown in FIG. 10C, or in one or the other of the Left Hepatic Artery (LPA) or Right Hepatic Artery (RHA).

Alternatively, De-Oxygenated blood rich in nutrition from the digestive system (Stomach-Large & small cologne-Pancreas-Spleen, etc.,) may be directed from the Inferior Vena Cava (IVC) through the Hepatic Portal Vein to Liver (MIS approach). “MIS” refers to Minimally-Invasive Surgery which may be required to install one or more balloon catheters due to complicated vasculature or inaccessible target sites. Occlusion and introduction of treated blood and payload may be via MIS at the Hepatic Portal Vein (HPV), or in one or the other of the Left Portal Vein (LPV) or Right Portal Vein (RPV). Depending on anatomy, the occlusion of Hepatic Veins could be done with one, two, or three catheters (one large one and three smaller ones are shown in FIG. 10A).

Finally, a combination of both of these techniques might be used. These options may apply to all 3 areas of treatment-Gene therapy, Oncology as well as solutions to treat Alzheimer patents.

### Isolation and Treatment of a Kidney

Although not shown, one or the other of the two kidneys may also be isolated using the systems and methods herein. For catheter access, common access sites are, femoral, jugular, and radial arteries/veins short of any cutdown. Typically, the supply line is introduced in a retrograde fashion (with the end of the catheter pointed opposite the direction of blood flow to the heart, and femoral access), while the return line is antegrade (with the end of the catheter pointed toward the direction of blood flow to the heart, and jugular access). However, the access sites may also change as well as the direction of access.

### Isolation and Treatment of the Pancreas

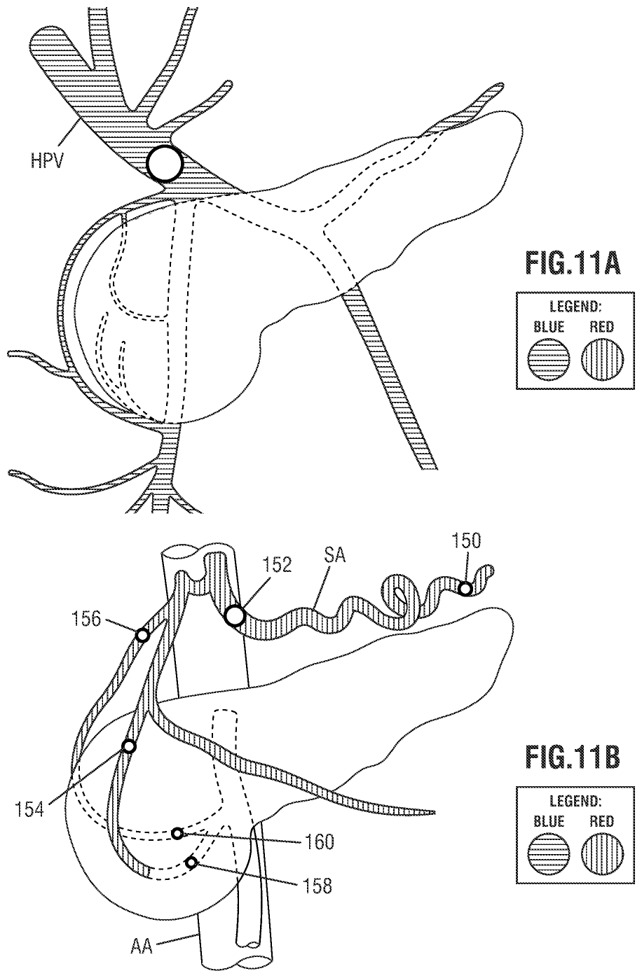

FIG. 11A is a schematic view of the pancreas and primary veins for collecting blood therefrom to the heart along with locations for positioning collection catheter balloons. Occlusion and retrieval of systemic blood and payload is usually done with inflation of balloon in the Hepatic Portal Vein (HPV) as shown. Alternatively, insertion of the collection catheter may be via MIS at Hepatic Portal Vein.

FIG. 11B shows the primary arteries for introducing blood to the pancreas along with locations for positioning supply catheter balloons. Occlusion and systemic supply to Spleen is indicated by a circle 150 toward the end of the Splenic Artery (SA). Occlusion and Introduction of treated blood and payload may also be via a larger end 152 of the Splenic Artery. Alternatively, treated blood and payload may be delivered at 154 to the Anterior Superior Pancreaticoduodenal Artery which may be done past the bifurcation of Gastroduodenal Artery and Right Gastroomental Artery. Alternatively, occlusion and introduction of treated blood and payload via Posterior Superior Pancreaticoduodenal Artery at 156. Also, occlusion and introduction of treated blood and payload may be through the Mesentric Artery at 158 at the bifurcation of Inferior Pancreaticoduodenal Artery near Anterior Inferior Pancreaticoduodenal Artery. Still further, occlusion and introduction of treated blood and payload could be via Mesentric Artery at 160 at the bifurcation of the Inferior Pancreaticoduodenal Artery near the Posterior Inferior Pancreaticoduodenal Artery. These locations are all shown by circles.

### Isolation and Treatment of the Spleen

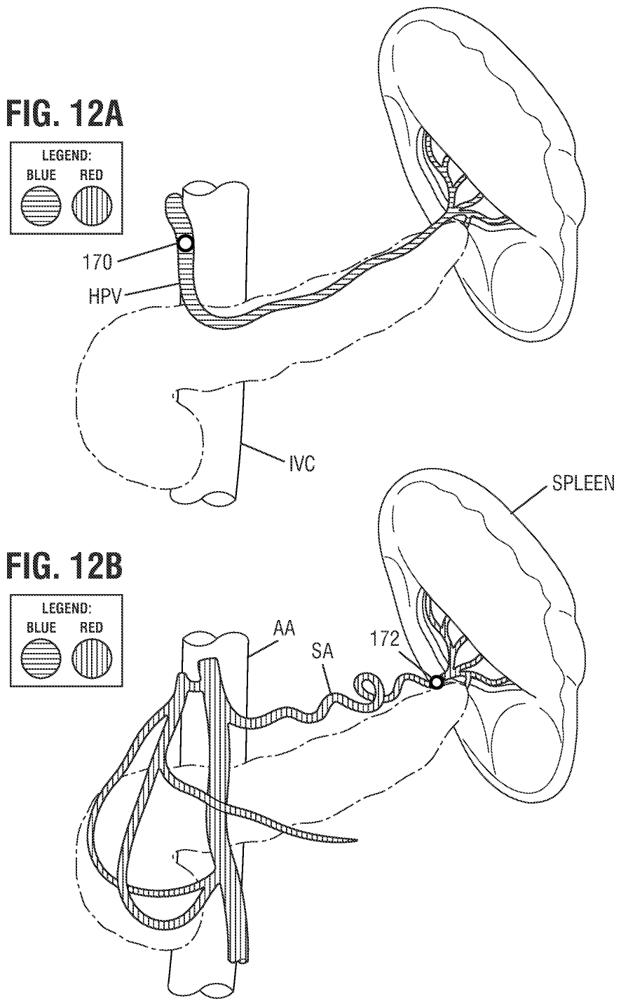

FIG. 12A is a schematic view of the spleen and primary veins for collecting blood therefrom to the heart along with locations for positioning collection catheter balloons. Occlusion and retrieval of systemic blood and payload is typically via MIS at Hepatic Portal Vein as shown at 170.

FIG. 12B shows the primary arteries for introducing blood to the spleen along with locations for positioning supply catheter balloons. Occlusion and introduction of treated blood and payload may be done via the Splenic Artery (SA) at the connection of the Spleen as shown at 172.

### Isolation and Treatment of the Head

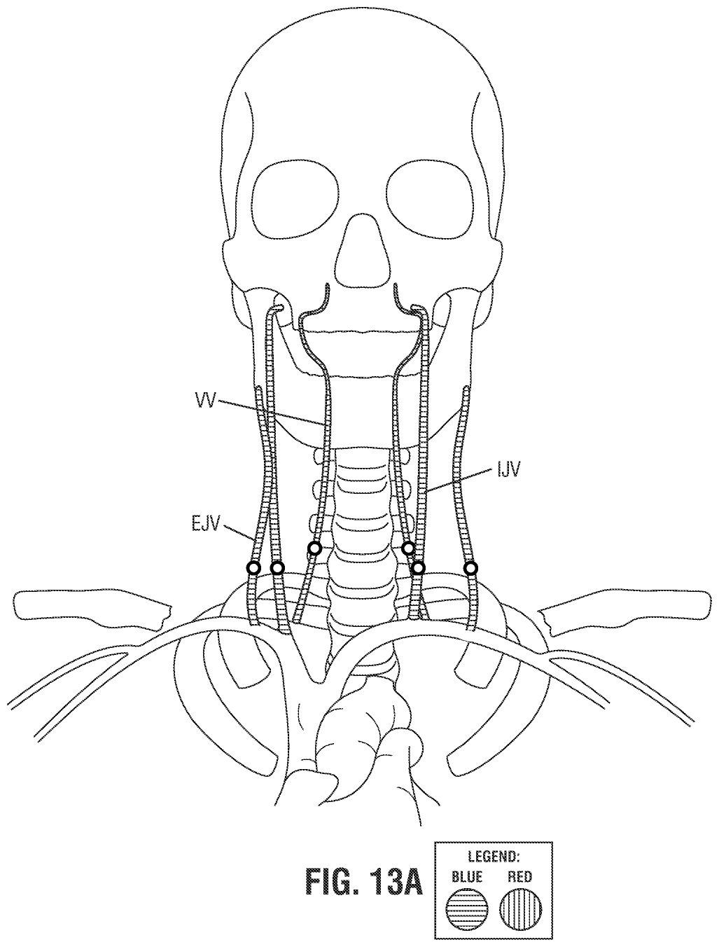

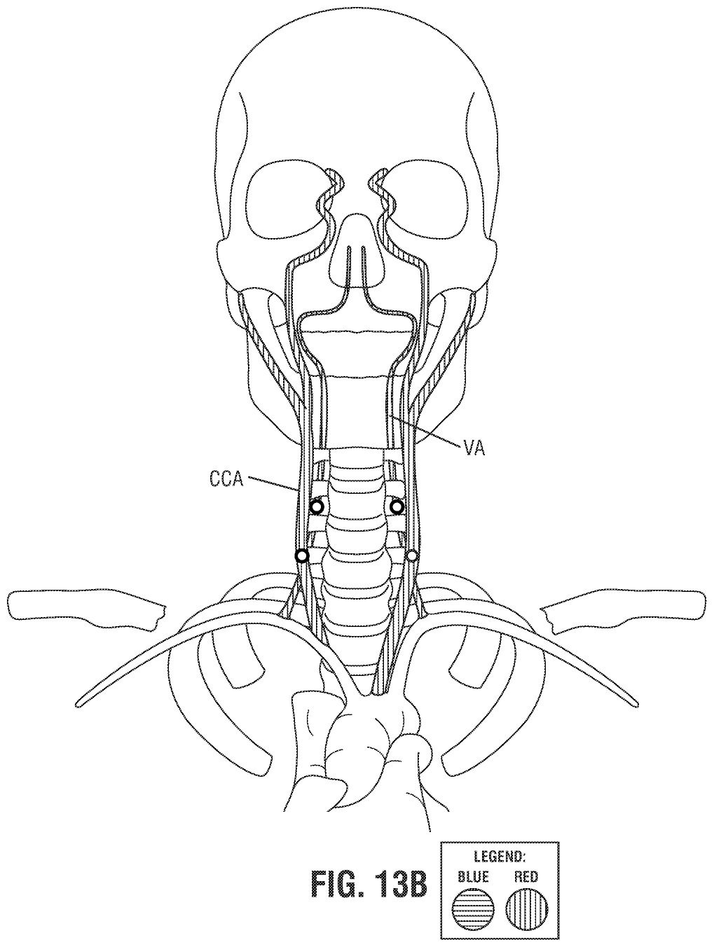

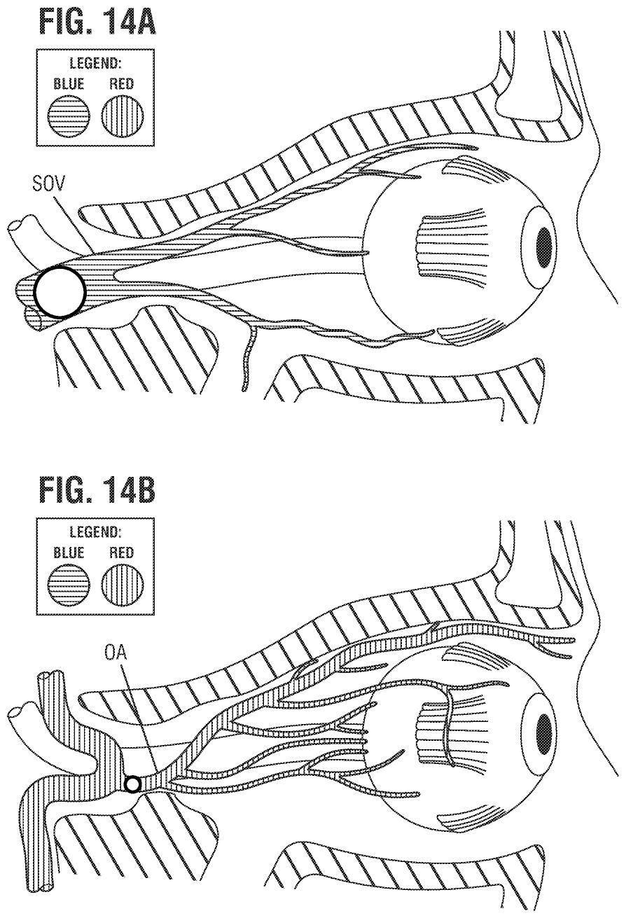

FIG. 13A is a schematic view of the head and primary veins for collecting blood therefrom to the heart along with locations for positioning collection catheter balloons. Occlusion and retrieval of systemic blood and payload may be via the External Jugular Veins (EJV), or via the Internal Jugular Veins (IJV) or Vertebral Veins (VV) (all either right or left), as shown by the circles representing placement of the distal ends of the return catheters.

FIG. 13B shows the primary arteries for introducing blood to the head along with locations for positioning supply catheter balloons. Occlusion and introduction of treated blood and payload may be via the Common Carotid Artery or the Vertebral Artery (either right or left), as shown by the circles representing placement of the distal ends of the supply catheters.

### Isolation and Treatment of the Eyes

FIG. 14A is a schematic view of one of the eyes and primary veins for collecting blood therefrom to the heart along with locations for positioning collection catheter balloons. Occlusion and retrieval of systemic blood and payload via Superior Ophthalmic Vein (SOV) (right or left).

FIG. 14B shows the primary arteries for introducing blood to the eye along with locations for positioning supply catheter balloons. Occlusion and introduction of treated blood and payload via Ophthalmic Artery (OA) (right or left).

### Isolation and Treatment of the Breasts

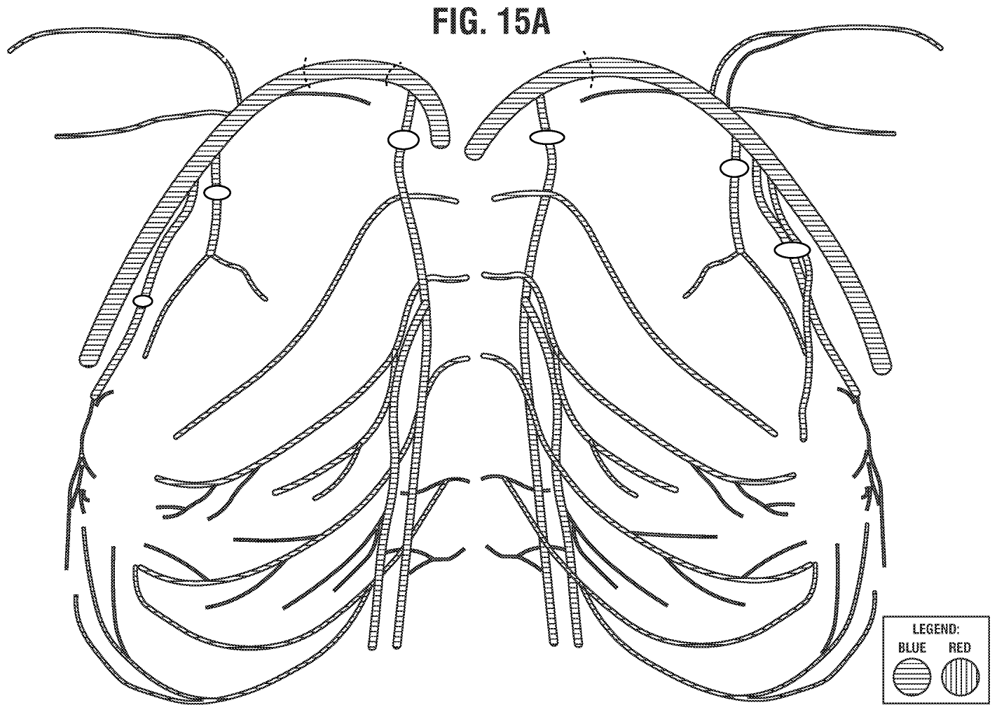

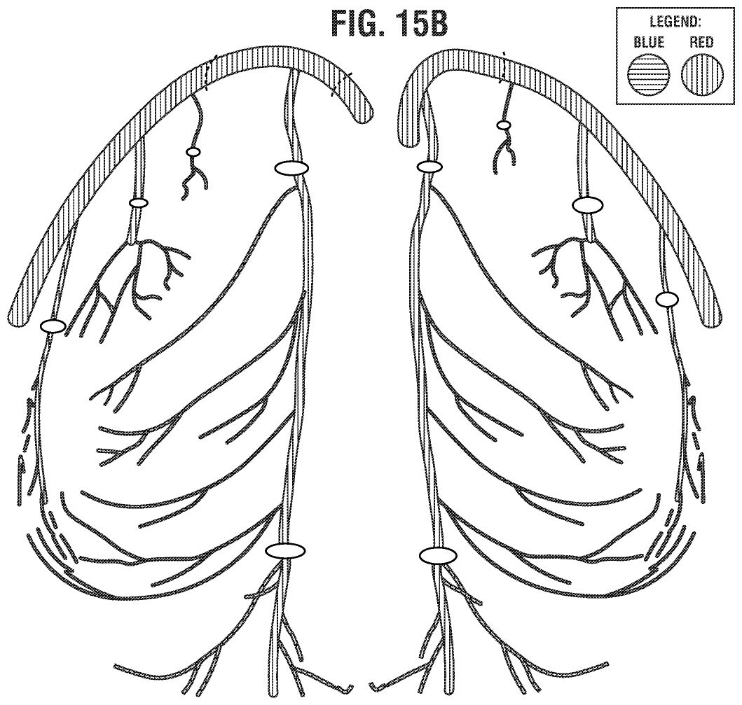

FIG. 15A is a schematic view of the breasts and primary veins for collecting blood therefrom to the heart along with locations for positioning collection catheter balloons. Occlusion and retrieval of systemic blood and payload are via a number of veins as shown.

FIG. 15B shows the primary arteries for introducing blood to the breasts along with locations for positioning supply catheter balloons. Occlusion and introduction of treated blood and payload are via a number of arteries as shown. The Superior Epigastric Arteries may be blocked and systemic flow permitted therethrough, as shown at the bottom, to prevent migration of treated solution to downstream locations.

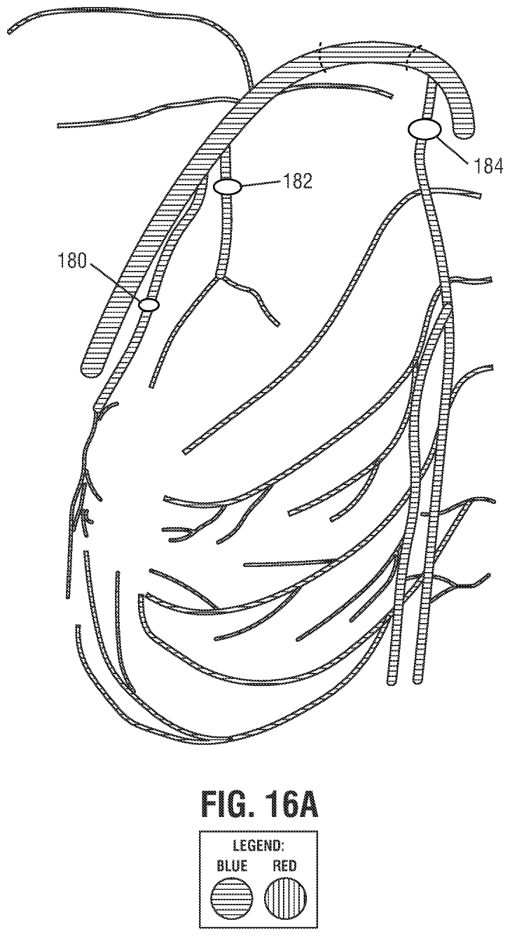

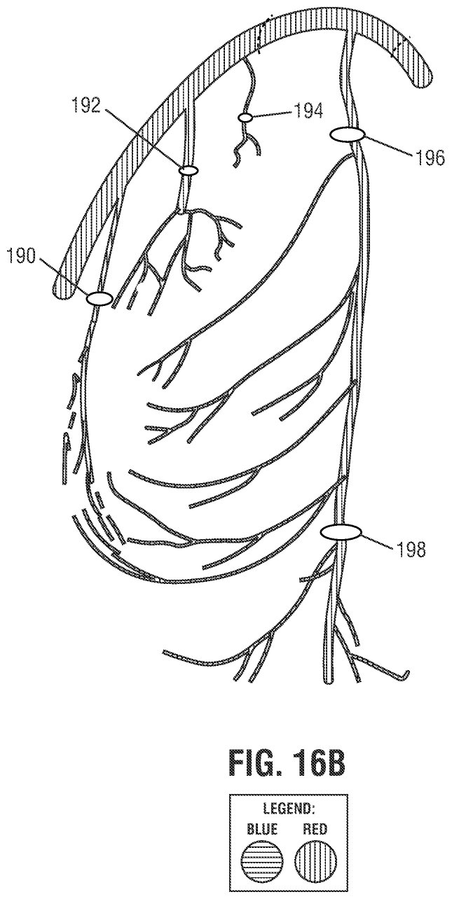

FIG. 16A is a schematic view of the right breast and primary veins for collecting blood therefrom to the heart along with locations for positioning collection catheter balloons. Occlusion and retrieval of treated blood supply and systemic blood supply may be via the Right Lateral Thoracic Vein from the Right Axillary Vein at 180, from the Lateral Mammary Branches of Lateral Vein Occlusion. Secondly, via the Right Thoracoacromial Vein from the Right Axillary Vein at 182. Further occlusion and retrieval of treated blood supply and systemic blood supply are via the Right Internal Thoracic Veins from the Right Brachiocephalic Vein, and from the Right Medial Mammary Branches of Internal Thoracic Veins at 184. The same may be done on the left side.

FIG. 16B shows the primary arteries for introducing blood to the right breast along with locations for positioning supply catheter balloons. Occlusion and introduction of treated blood supply may be via the Right Lateral Thoracic Artery from the Right Axillary Artery at 190, supplying the Lateral Mammary Branches of Lateral Artery. Or, via the Right Pectoral Branch of the Thoracoacromial Artery from the Right Axillary Artery at 192. Optionally, depending on imaging, occlusion and introduction of treated blood supply are via the Right Superior Thoracic Artery from the Right Subclavian Artery at 194. Fourthly, occlusion and introduction of treated blood supply may be via the Right Internal Thoracic Artery from the Brachiocephalic Trunk at 196, supplying the Medial Mammary Branches of Internal Thoracic Artery. Finally, treatment may be via the Right Superior Epigastic Artery as at 198. The same may be done on the left side.

### Isolation and Treatment of the Female Reproductive Systems

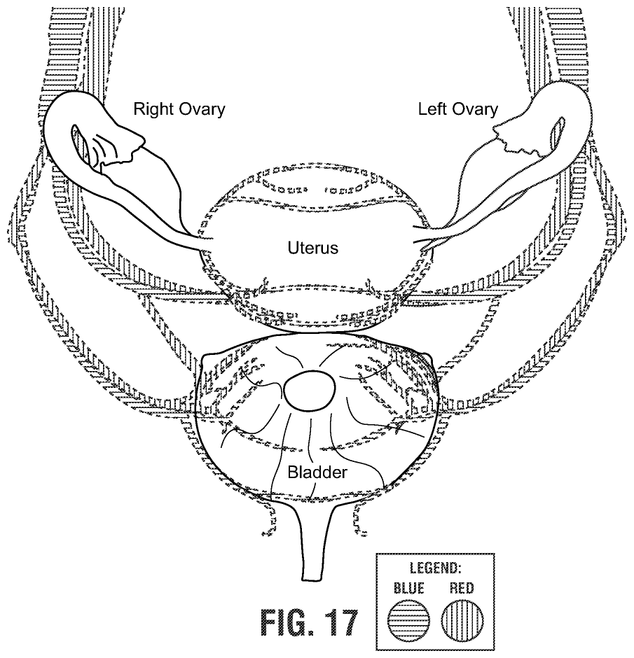

FIG. 17 is a schematic view of the female uterus, bladder and reproductive system along with an overview of the blood vessels therein.

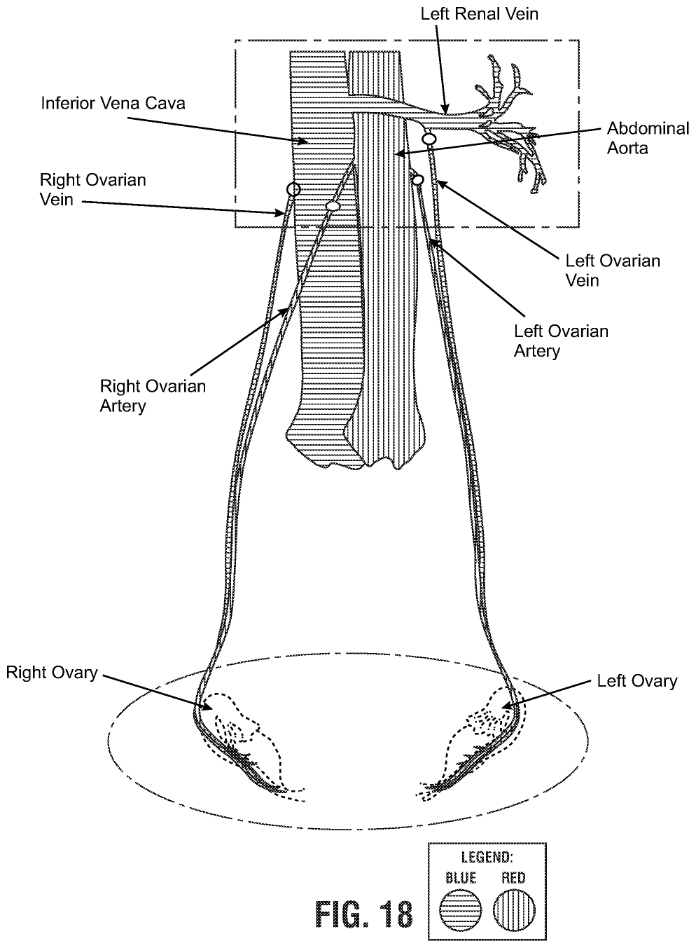

FIG. 18 is a diagram of the blood flow to the female ovaries along with locations for positioning both collection and supply catheter balloons as indicated, and FIG. 18A is a detail thereof. The ability to place the supply and return catheters in one or the other, or both of the ovaries, enables targeting the treatment solution to just where it is needed. For instance, the supply can be delivered to just the right Ovarian Artery and then retrieved from the Right Ovarian Vein and re-oxygenated to complete the isolated circuit. The catheters may be delivered through the Abdominal Aorta and Inferior Vena Cava, respectively.

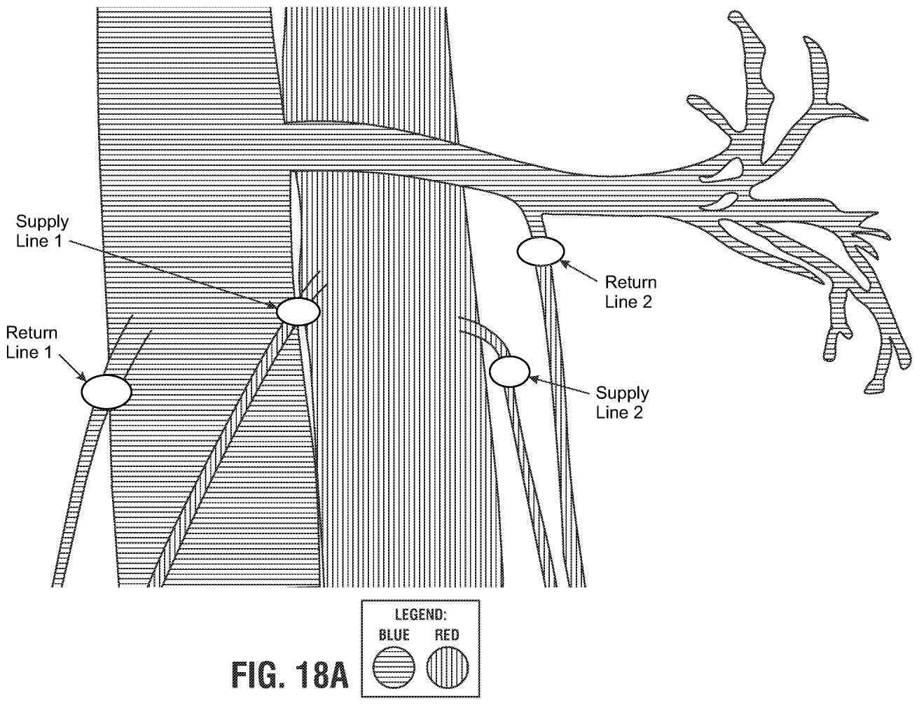

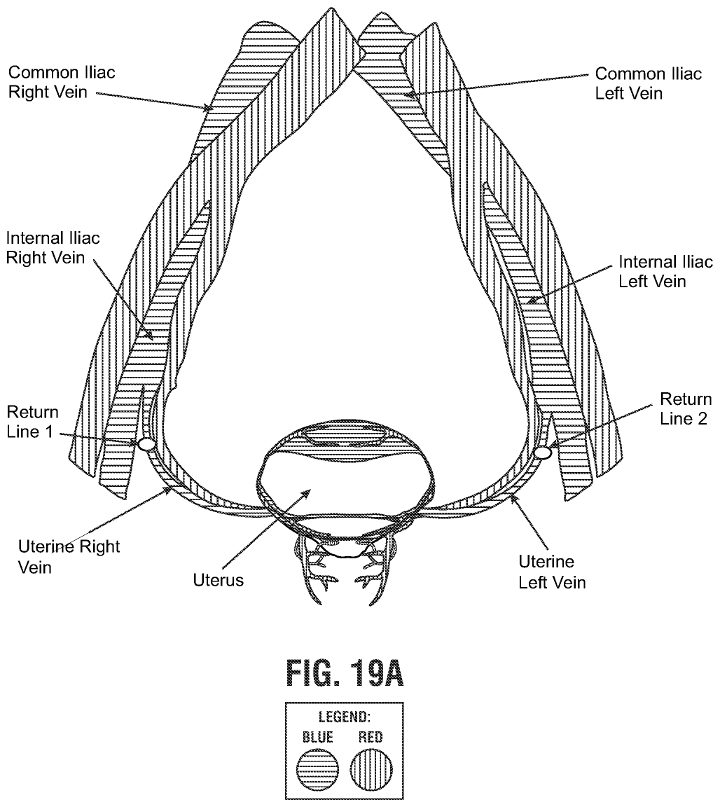

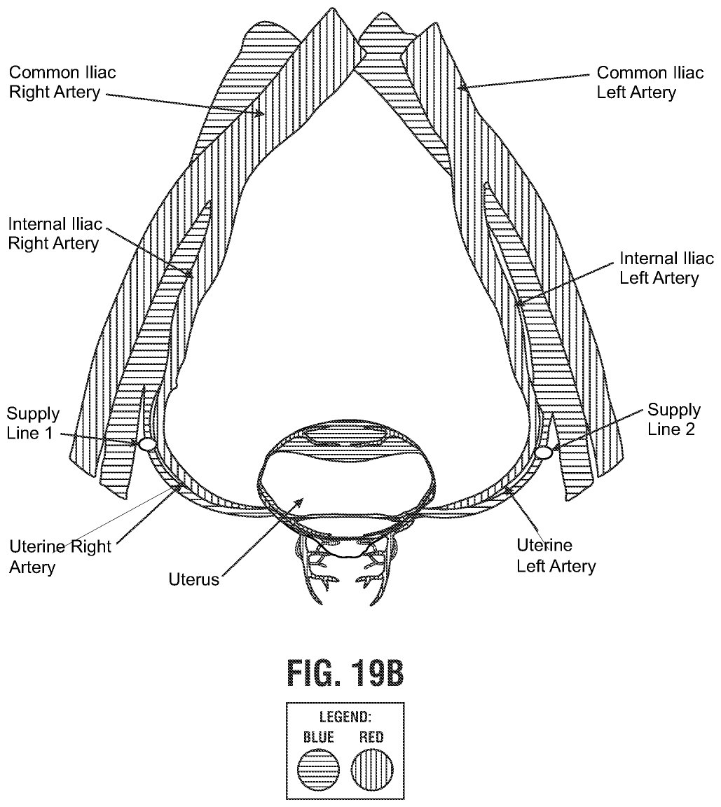

FIG. 19A is a schematic view of the female uterus and primary veins for collecting blood therefrom to the heart along with locations for positioning collection catheter balloons, as indicated. Occlusion and Retrieval of treated blood and payload may be via the Uterine Right Vein may be via access from the Internal Iliac Right Vein, and/or via Uterine Left Vein via access from the Internal Iliac Left Vein, as shown. FIG. 19B shows the primary arteries for introducing blood to the female uterus along with locations for positioning supply catheter balloons as shown. Conversely, occlusion and introduction of treated blood and payload may be via the Uterine Right Artery via access from the Internal Iliac Right Artery, or via the Uterine Left Artery via access from the Internal Iliac Left Artery.

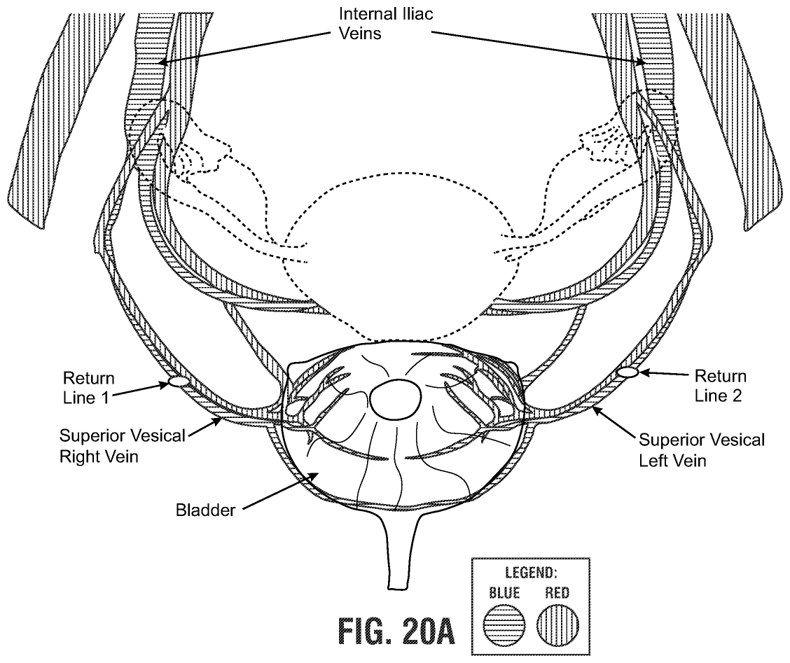

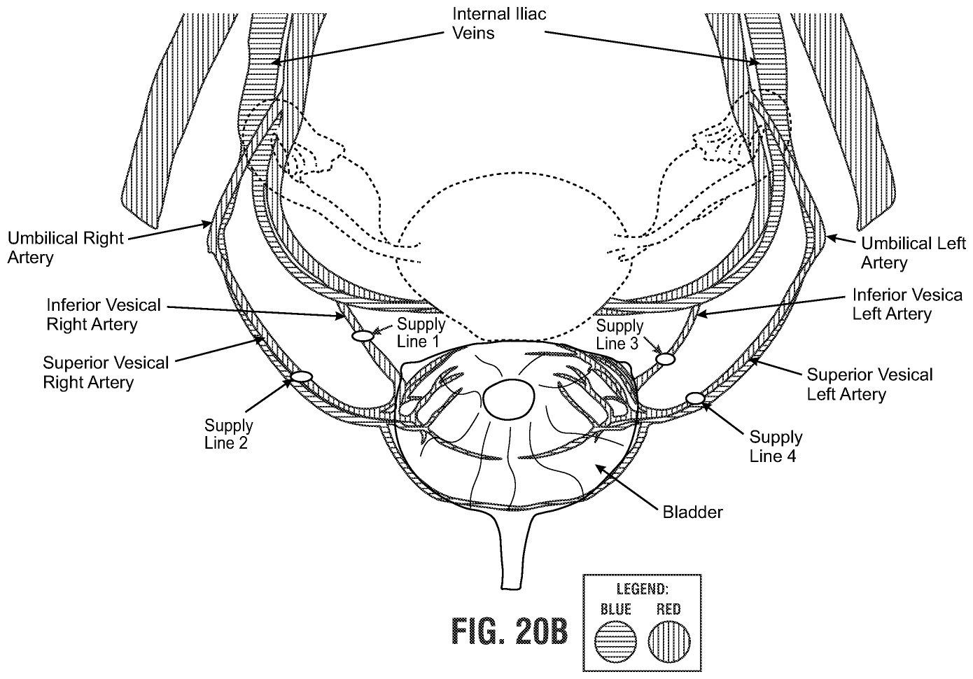

FIG. 20A is a schematic view of the female bladder and primary veins for collecting blood therefrom to the heart along with locations for positioning collection catheter balloons, as indicated. Occlusion and Retrieval of treated blood and payload may be via the Superior Vesical Right Vein with access from the Internal Iliac Right Vein, and/or via the Superior Vesical Left Vein with access from the Internal Iliac Left Vein. FIG. 20B shows the primary arteries for introducing blood to the female bladder along with locations for positioning supply catheter balloons as indicated. In various configurations, occlusion and introduction of treated blood and payload may be via the Inferior Vesical Right Artery with access from the Internal Iliac Right Artery, via the Superior Vesical Right Artery with access from the Internal Iliac Right Artery, via Inferior Vesical Left Artery with access from Umbilical Right Artery, or via the Superior Vesical Left Artery with access from the Umbilical Left Artery.

Drugs approved for use in the United States for the treatment of cervical cancer include bevacizumab, bleomycin, and topotecan hydrochloride, and the combination gemcitabine-cisplatin. Uterine cancer of endometrial origin may be treated with, for example, megestrol acetate.

### Isolation and Treatment of the Male Reproductive Systems

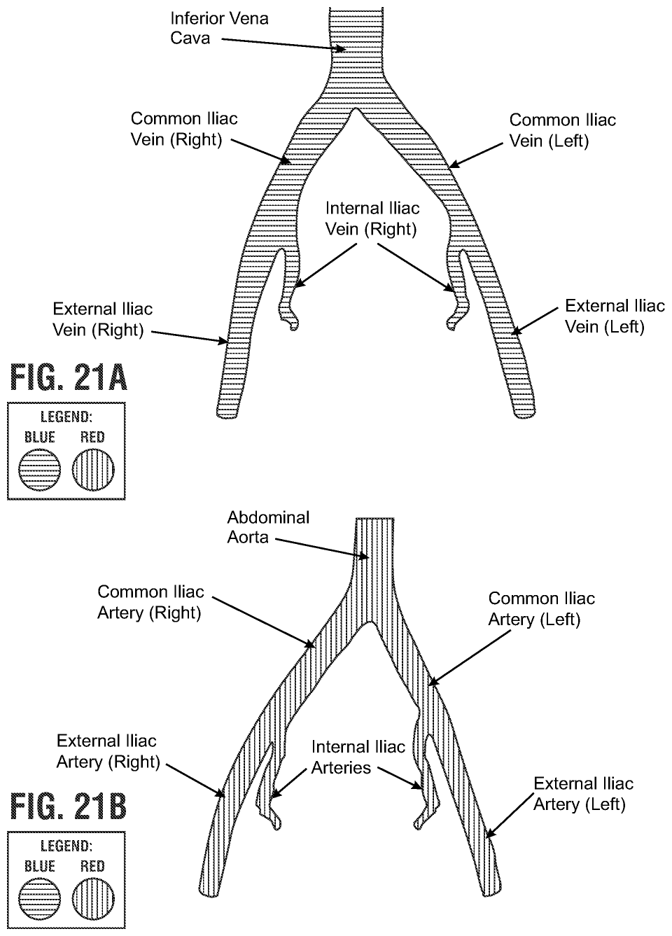

FIG. 21A is a diagram of the large veins located in the male crotch region, and FIG. 21B is a diagram of the large arteries therein.

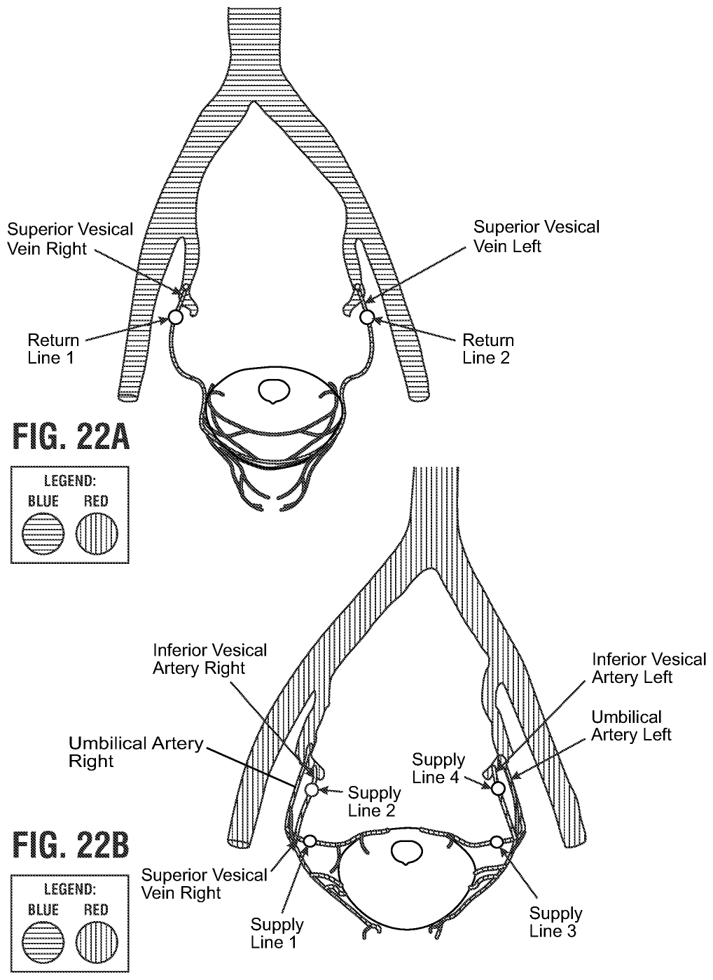

FIG. 22A is a schematic view of the male bladder and primary veins for collecting blood therefrom to the heart along with locations for positioning collection catheter balloons, as shown. Occlusion and retrieval of therapeutic treated blood may be via the Right Superior Vesical Vein at the Right Internal Iliac Vein, which drains from Vesical Venous Plexus and Prostatic Venous Plexus. Alternatively or in addition, occlusion and retrieval may be via the Left Superior Vesical Vein at the Left Internal Iliac Vein, which drains from Vesical Venous Plexus and Prostatic Venous Plexus.

FIG. 22B shows the primary arteries for introducing blood to the male bladder along with locations for positioning supply catheter balloons as indicated. Occlusion and supply of therapeutic treated blood may be via the Right Superior Vesical Arteries from the Right Umbilical Artery extending from the Right Internal Iliac Artery, or via the Right Inferior Vesical Artery extending from the Right Internal Iliac Artery, or via the Left Superior Vesical Arteries from the Left Umbilical Artery extending from the Left Internal Iliac Artery, or also via the Left Inferior Vesical Artery extending from the Left Internal Iliac Artery.

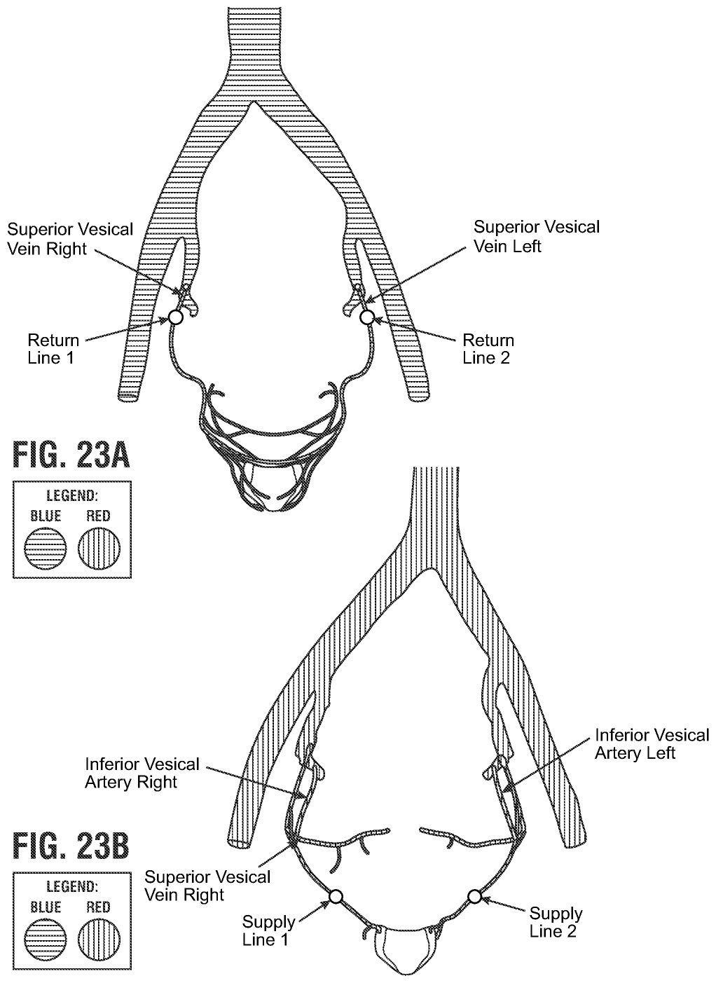

FIG. 23A is a schematic view of the male prostate and primary veins for collecting blood therefrom to the heart along with locations for positioning collection catheter balloons, as indicated. Occlusion and retrieval of therapeutic treated blood may be via the Right Superior Vesical Vein at the Right Internal Iliac Vein, which drains from Vesical Venous Plexus and Prostatic Venous Plexus, or via the Left Superior Vesical Vein at the Left Internal Iliac Vein which drains from Vesical Venous Plexus and Prostatic Venous Plexus.

FIG. 23B shows the primary arteries for introducing blood to the male prostate along with locations for positioning supply catheter balloons as shown. Occlusion and supply of therapeutic treated blood may be via the Right Inferior Vesical Artery extending from the Right Internal Iliac Artery. Alternatively, occlusion and supply of therapeutic treated blood may be via the Left Inferior Vesical Artery extending from the Left Internal Iliac Artery. Where both the Bladder and Prostate share these feeding Arteries the Catheter is desirably placed closer to the Prostate to isolate the treatment.

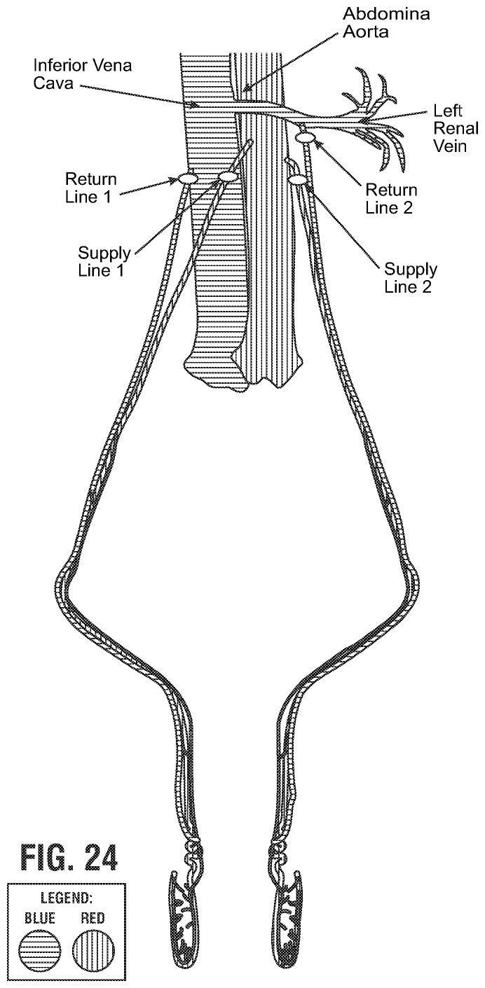

FIG. 24 is a diagram of the blood flow to the male testicles along with locations for positioning both collection and supply catheter balloons as shown. Occlusion and retrieval of therapeutic treated blood may be via the Right or Left Testicular Vein from the bifurcation of Abdominal Aorta. Retrieving from Right or Left Pampiniform Plexus and Right or Left Testicular Pampiniform Plexus. Occlusion and supply of therapeutic treated blood may be via the Right or Left Testicular Artery from the bifurcation of Abdominal Aorta.

### Other Oncology Applications

For specific areas of the body, the isolation systems disclosed herein can target and/or isolate just a tumor or tumors with perfusion of higher concentration of the therapeutic agent for more effectiveness. For instance, a common chemotherapy drug for brain tumors is temozolomide, and a combination of drugs called procarbazine, lomustine and vincristine (PCV).

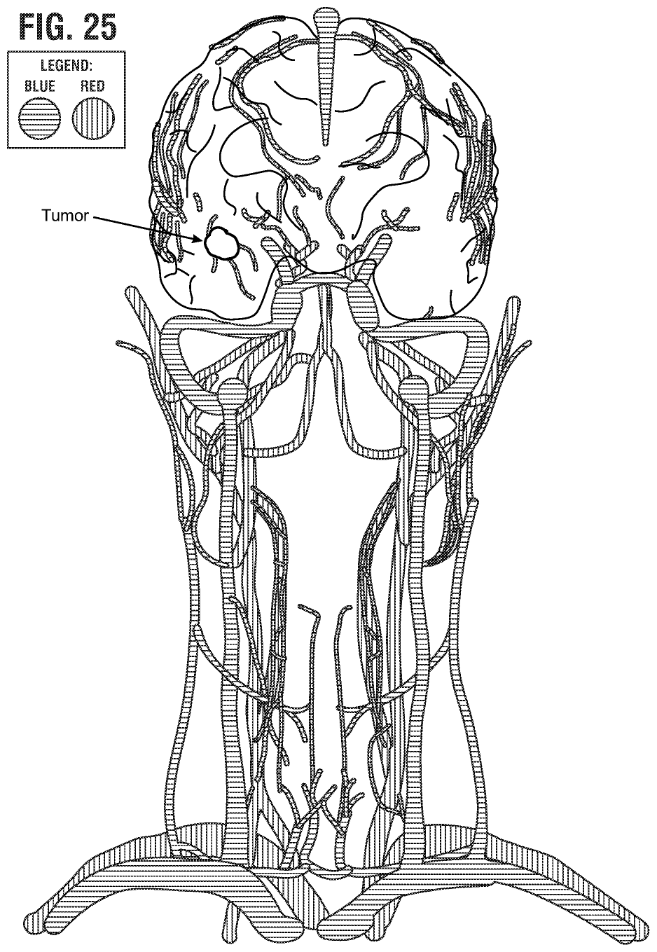

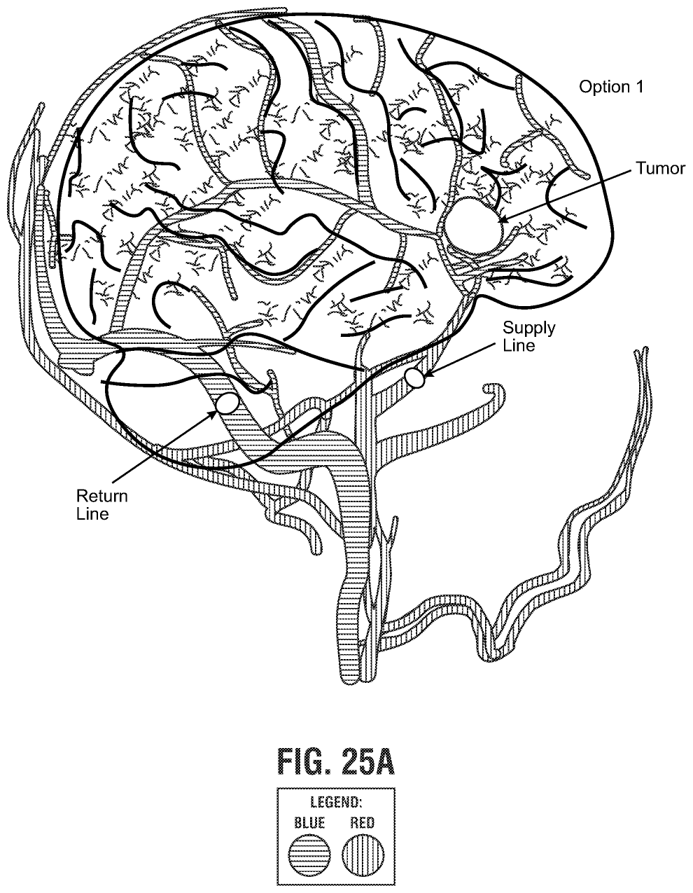

FIG. 25 is a schematic view of the upper thorax, neck and brain along with an overview of the blood vessels therein, along with a typical position of a brain tumor.

FIG. 25A is a schematic view of the brain primary vessels for collecting and introducing blood thereto along with locations for positioning collection and supply catheter balloons to isolate and treat the tumor. The method includes occlusion and retrieval of drug agent and systematic mixed blood from body and then occlusion and targeted supply of oncology drugs at the site of tumor or close proximity to tumor location.

FIG. 25B is a schematic view of the brain primary veins for collecting and introducing blood thereto along with alternative locations for positioning collection and supply catheter balloons to isolate and treat the tumor. Instead of supplying the return blood and drug to nearby vessels, the treatment is a controlled supply of oncology drug through a catheter supply line 200 at low pressures at the site of tumor (with or without occlusion).

Because the vessels in the cranial cavity are substantially smaller capillaries, and it is desirable to target selected tumors to treat, for oncology & Alzheimer applications, a balloon at the distal end of the catheter may be eliminated to allow the catheter to go more distally in the smaller vessels. Where the arterial vessel is a capillary, the catheter supply line 200 is sized to fit within and occlude the capillary. Also, the pressure and the flow will be reduced while supplying the therapeutic agent to eliminate retrograde blood flow.

### Isolation and Treatment of the Colon

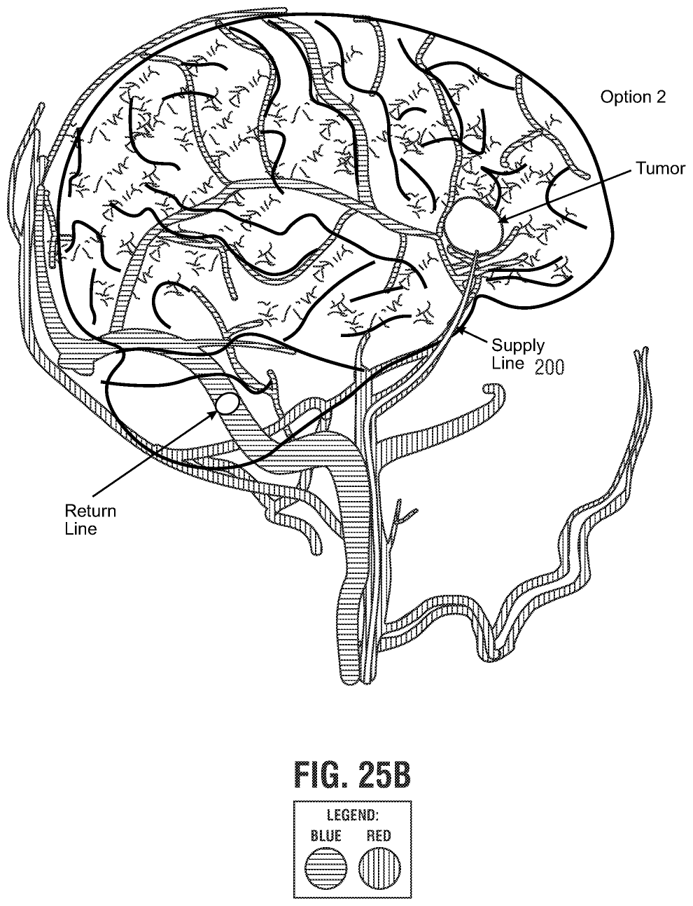

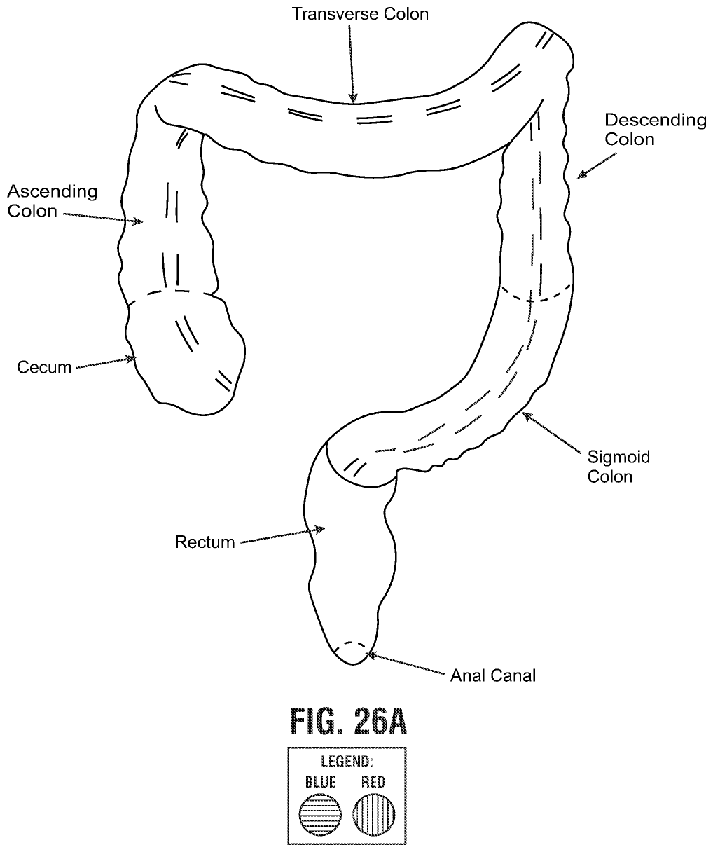

FIG. 26A is a schematic view of the colon with labels for different sections thereof, and FIG. 26B shows an overview of the blood vessels therein.

FIG. 27A is a schematic view of the colon and primary veins for collecting blood therefrom to the heart, and FIG. 27B shows the primary veins isolated along with locations for positioning collection catheter balloons. The method involves first MIS at hepatic portal vein for retrieval access at RETURN LINE 1. A further step at RETURN LINE 2 includes occlusion and retrieval of treated blood and payload via inferior mesenteric vein via access from the splenic vein. This should aid in draining the left colic, sigmoid, middle colic, and anorectal veins. AT RETURN LINE 3 occlusion and retrieval of treated blood and payload via superior mesenteric vein is done via access from the inferior vena cava. This could take place before the bifurcation of middle colic vein to aid in draining right colic, ileocolic, and middle colic veins. RETURN LINE 4 represents occlusion and retrieval of treated blood and payload via middle anorectal right vein access from the internal right iliac vein, which should aid in draining the middle anorectal right vein and joining anorectal veins. Finally, occlusion and retrieval of treated blood and payload via middle anorectal left vein access from the internal left iliac vein is seen at RETURN LINE 5.

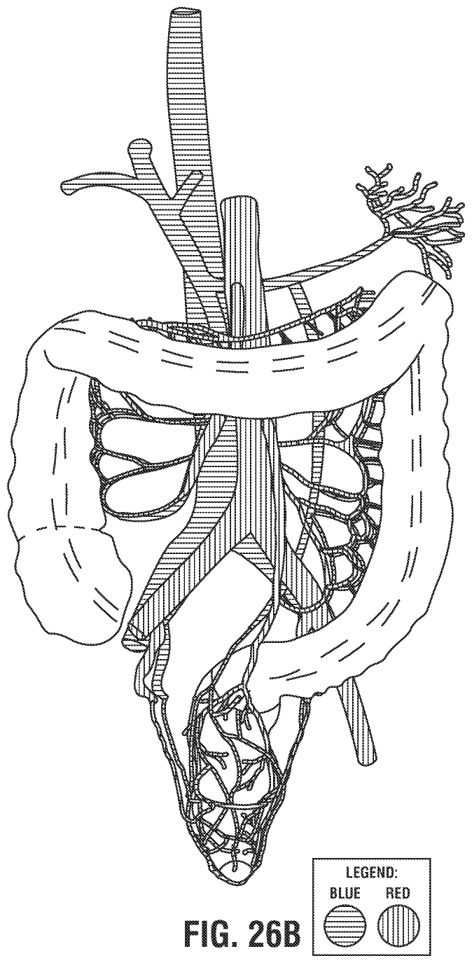

FIG. 28A is a schematic view of the colon and primary arteries for introducing blood from the heart, and FIG. 28B shows the primary arteries isolated along with locations for positioning supply catheter balloons. SUPPLY LINE 1 performs occlusion and introduction of treated blood and payload via inferior mesenteric artery via access from the abdominal aorta, supplying the following arteries: left colic, sigmoid, descending marginal and superior anorectal arteries. SUPPLY LINE 2 adds occlusion and introduction of treated blood and payload via superior mesenteric artery via access from the abdominal aorta. Balloon placement should be proximal of middle colic artery bifurcation from superior mesenteric artery, supplying the following arteries: ascending marginal, transversing marginal, middle colic, right colic, left colic, and ileocolic arteries. Occlusion and introduction of treated blood and payload via middle right and left anorectal arteries via access from the internal right and left iliac arteries, supplying the rectum, may be done by SUPPLY LINES 3 and 4, respectively.

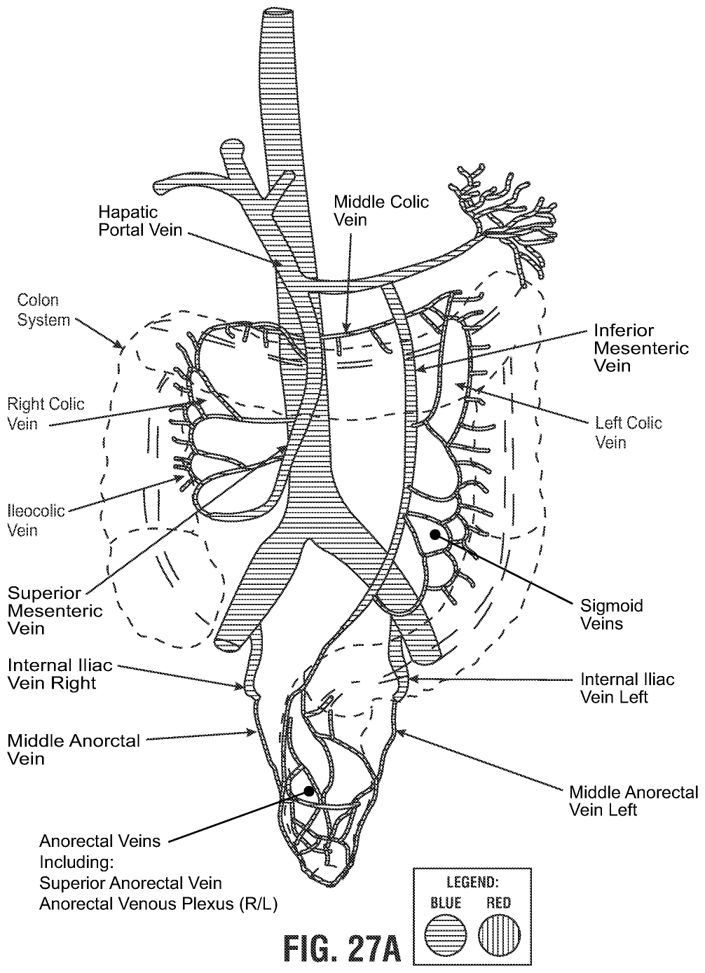

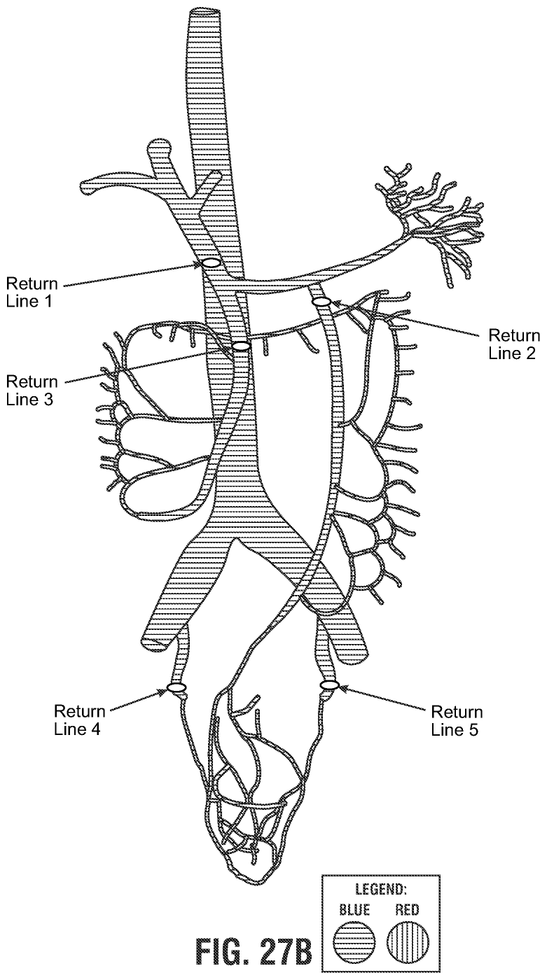

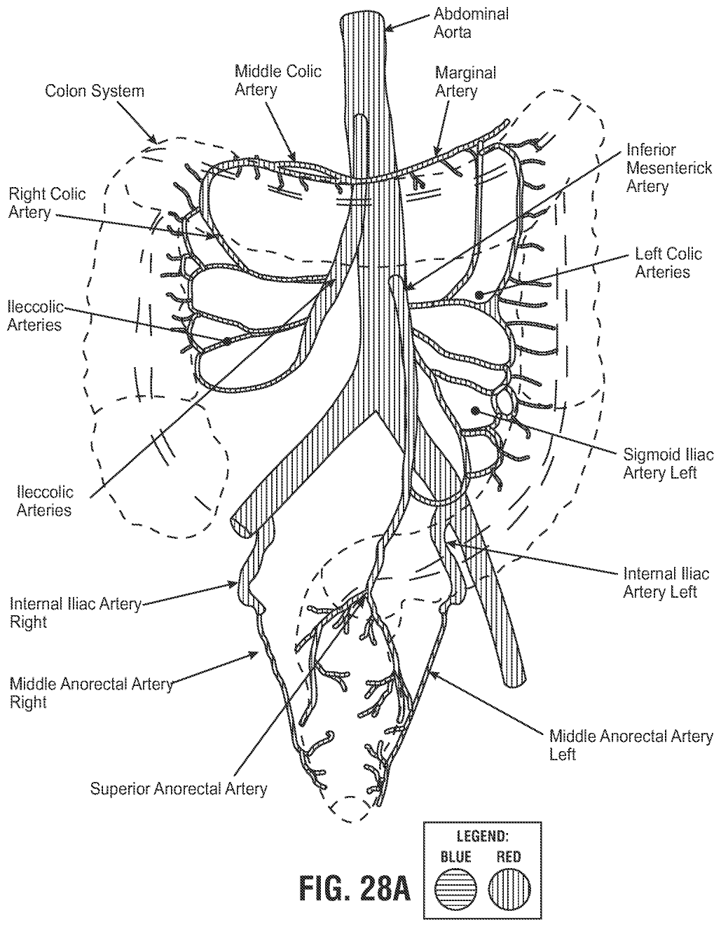

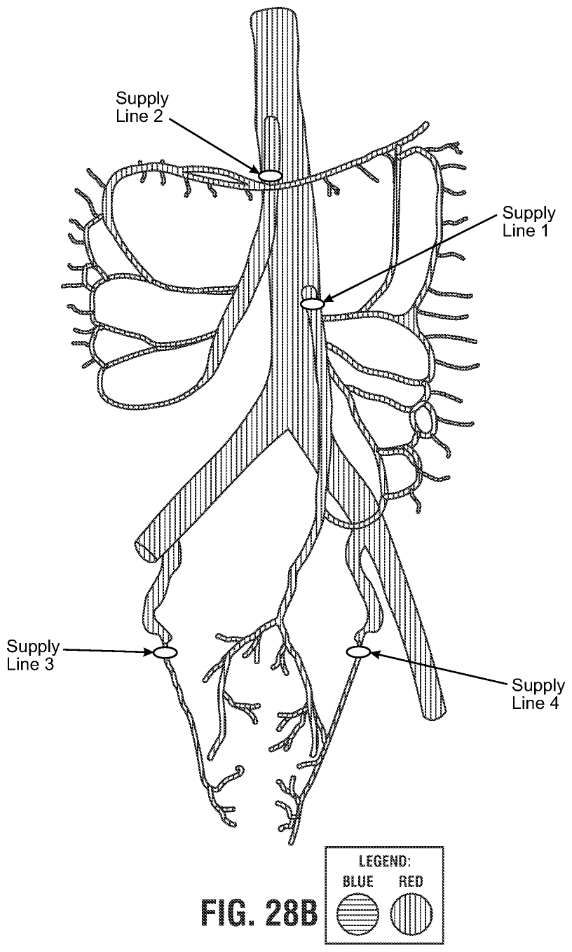

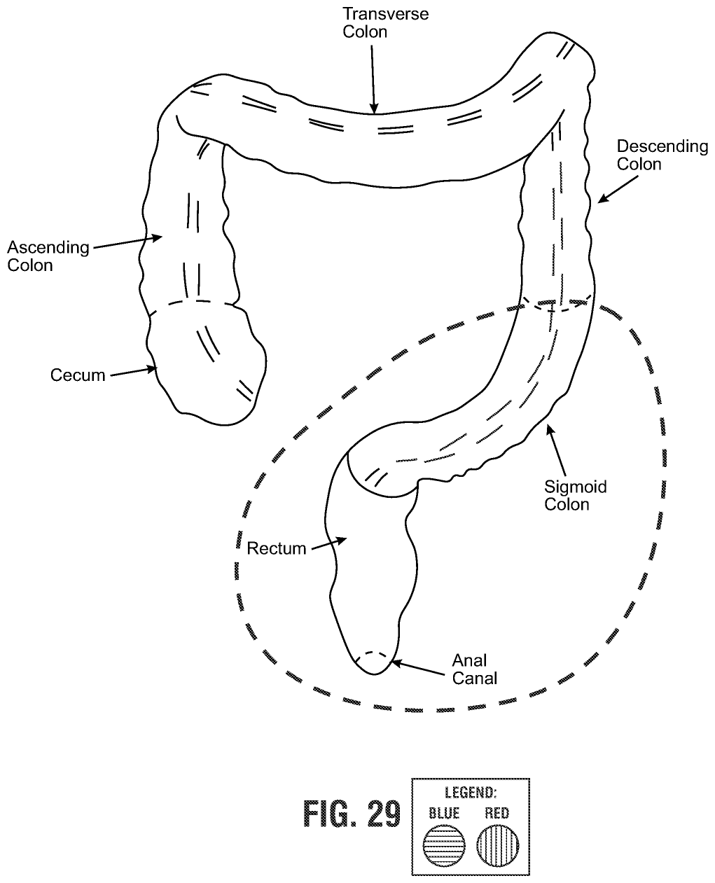

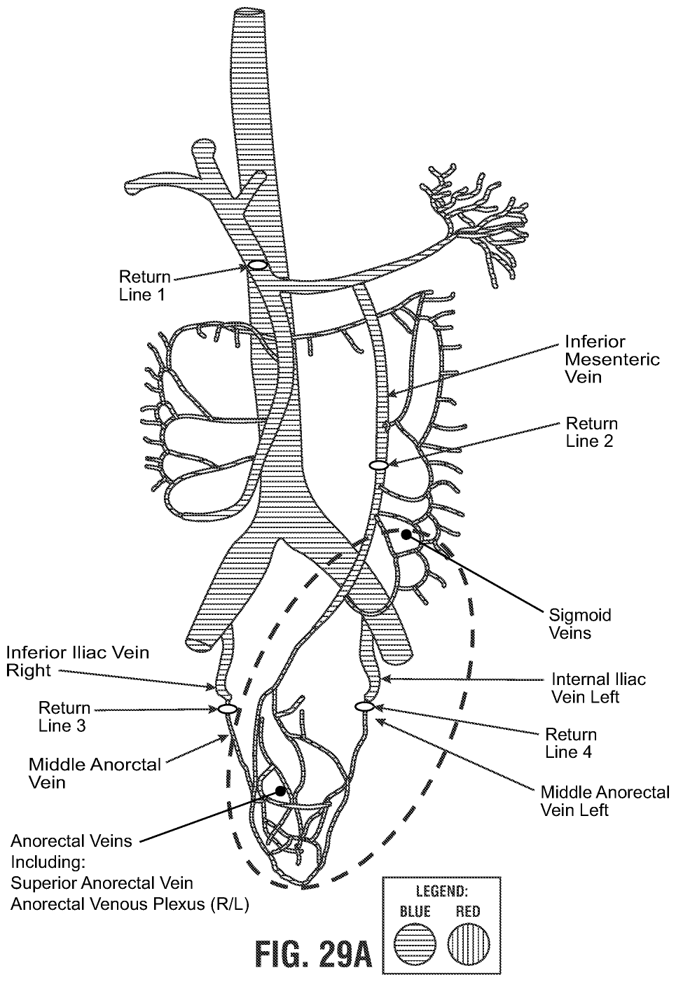

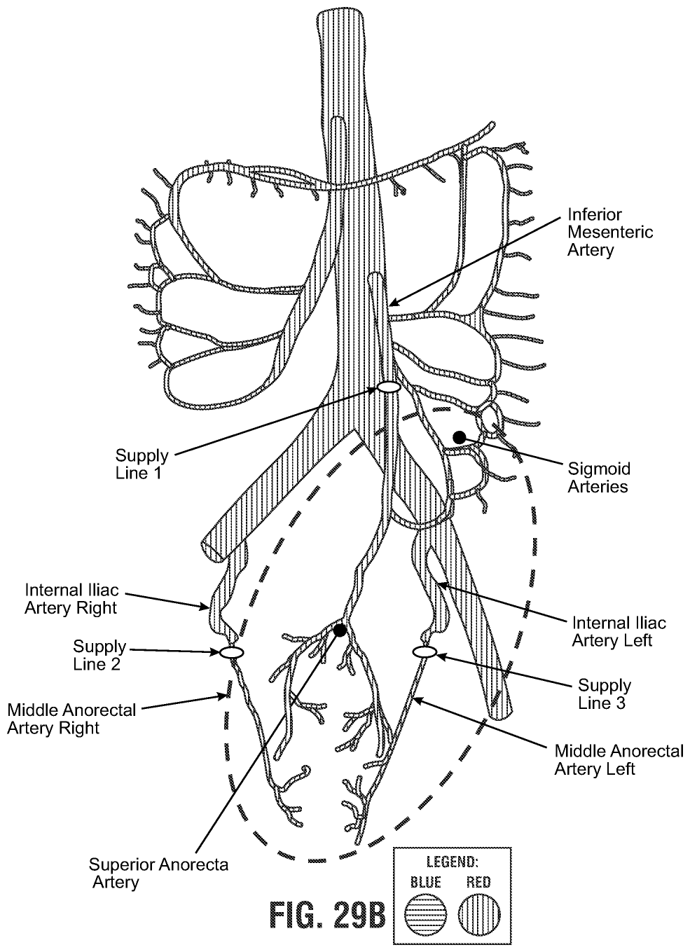

FIG. 29 is another schematic view of the colon with labels for different sections thereof and the colorectal region outlined.

Drugs approved for use in treating colon cancer in the United States include bevacizumab, irinotecan hydrochloride, capecitabine, cetuximab, ramucirumab, oxaliplatin, 5-FU, fluorouracil, leucovorin calcium, trifluridine, tipiracil hydrochloride, oxaliplatin, panitumumab, ramucirumab, regorafenib, ziv-aflibercept and the combinations capox, folfiri-bevacizumab, folfiri-cetuximab, FU-LV, xeliri and xelox.

FIG. 29A is a schematic view of the primary veins in the colorectal region along with locations for positioning collection catheter balloons to isolate and treat diseases therein. RETURN LINE 1 represents MIS at the hepatic portal vein for retrieval access. RETURN LINE 2 performs occlusion and retrieval of treated blood and payload via inferior mesenteric vein via access from the splenic vein. Occlusion should happen deep in the inferior mesenteric vein distal of sigmoid bifurcation, which should aid in draining the sigmoid and anorectal veins. Occlusion and retrieval of treated blood and payload via middle anorectal right vein access from the internal right iliac vein, which should aid in draining the middle anorectal right vein and joining anorectal veins, is done by RETURN LINE 3. Finally, RETURN LINE 4 adds occlusion and retrieval of treated blood and payload via middle anorectal left vein access from the internal left iliac vein, also aiding in draining the middle anorectal left vein and joining anorectal veins.

FIG. 29B is a schematic view of the primary arteries in the colorectal region along with locations for positioning supply catheter balloons to isolate and treat diseases therein. Occlusion and introduction of treated blood and payload via inferior mesenteric artery via access from the abdominal aorta is represented by SUPPLY LINE 1. Occlusion should happen deep in the inferior mesenteric artery distal of sigmoid bifurcation supplying the following arteries: sigmoid, descending marginal and superior anorectal arteries. SUPPLY LINES 2 and 3, respectively, occlude and introduce treated blood and payload via middle right and left anorectal arteries via access from the internal right and left iliac arteries, supplying the rectum.

### Isolation and Treatment of the Stomach

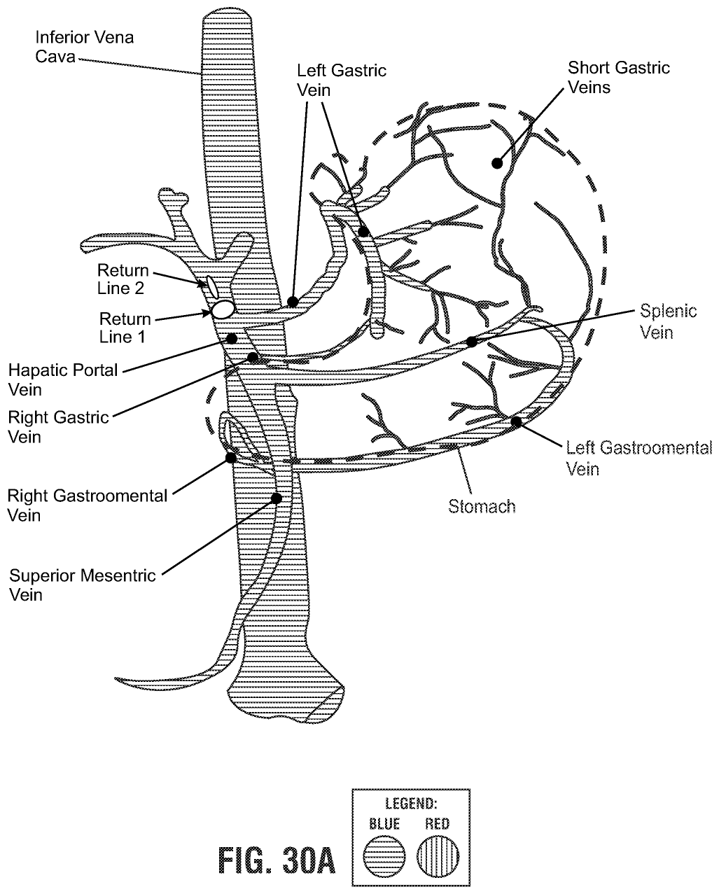

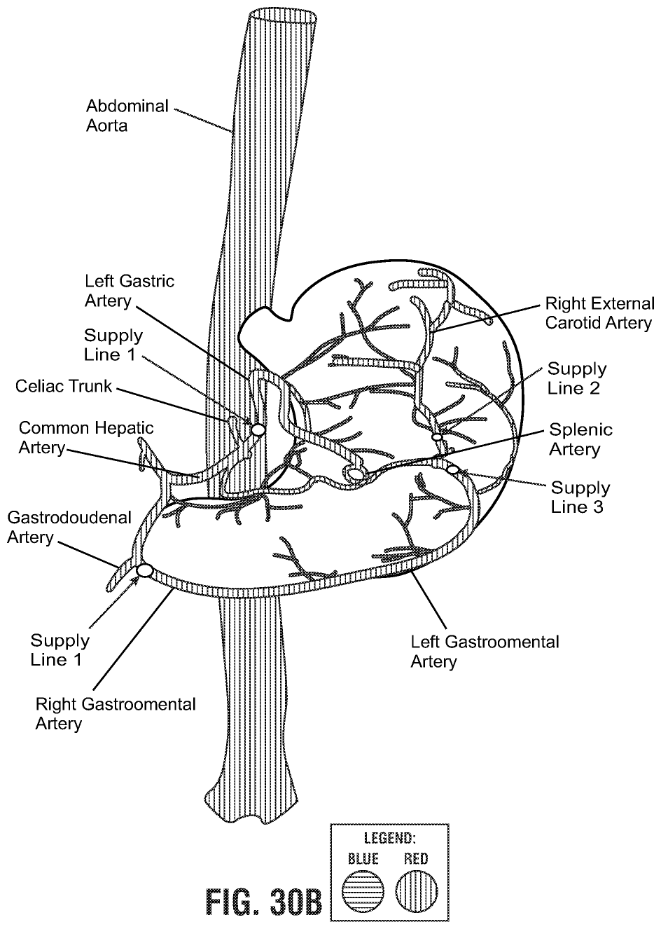

FIG. 30A is a schematic view of the primary veins in the stomach region along with locations for positioning collection catheter balloons to isolate and treat diseases therein. RETURN LINE 1 represents MIS at the hepatic portal vein for retrieval access. RETURN LINE 2 performs occlusion and retrieval of treated blood and payload via hepatic portal vein. Access at the hepatic portal vein will allow payload draining from left gastric vein, right gastric vein, right gastroomental vein, left gastroomental vein, splenic vein, and short gastric veins. The goal here is to prevent payload from being filtered in the liver.

FIG. 30B is a schematic view of the primary arteries in the stomach region along with locations for positioning supply catheter balloons to isolate and treat diseases therein. SUPPLY LINE 1 performs occlusion and introduction of treated blood and payload via left gastric artery being accessed from the celiac trunk, bifurcating from the abdominal aorta. Occlusion and introduction of treated blood and payload via short gastric arteries being accessed from the bifurcation of the splenic arteries is done by SUPPLY LINE 2, while occlusion and introduction of treated blood and payload via left gastroomental artery being accessed from the bifurcation of the splenic arteries is the job of SUPPLY LINE 3. Finally, SUPPLY LINE 4 occludes and introduces treated blood and payload via right gastroomental artery being accessed from the gastroduodenal artery which connects to the common hepatic and celiac trunk arteries which, in turn, bifurcate from the abdominal aorta.

### Isolation and Treatment of the Esophagus

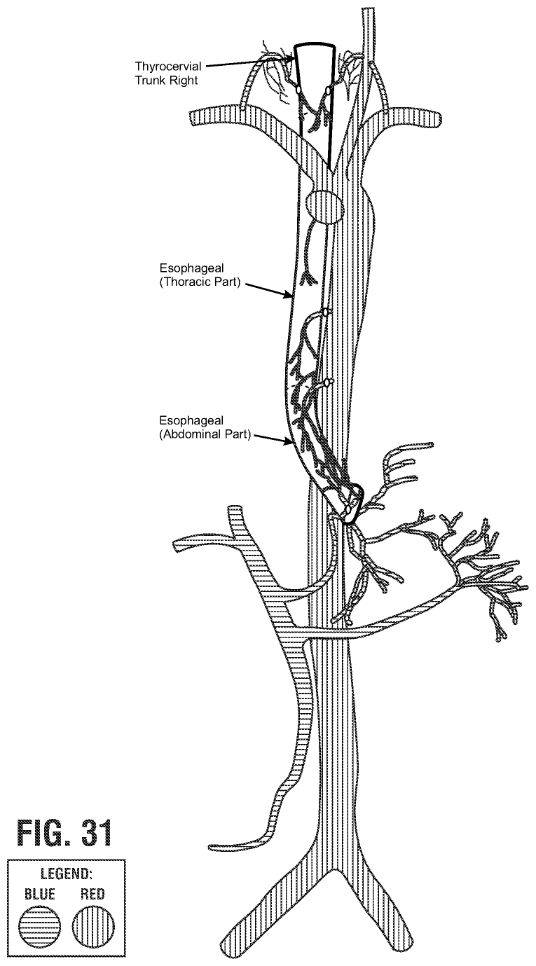

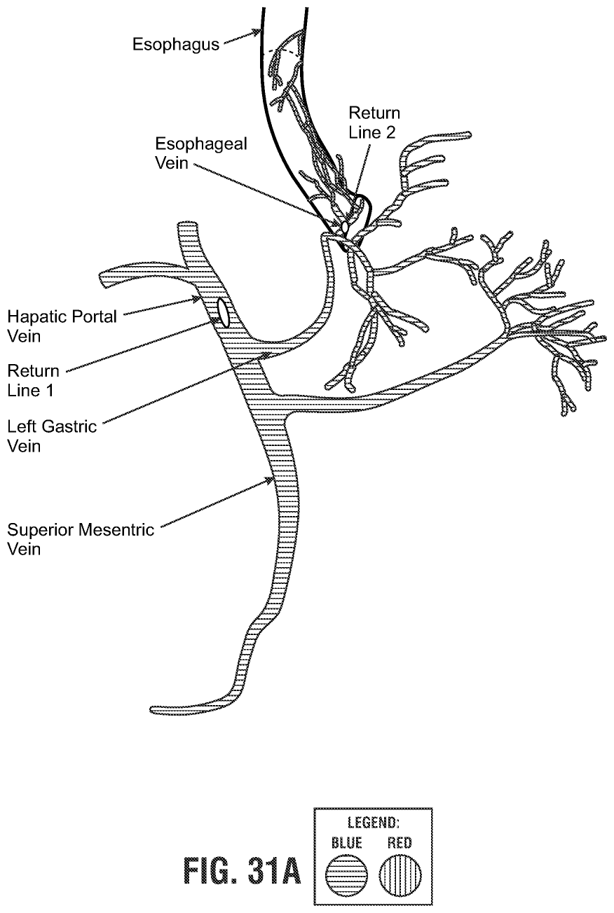

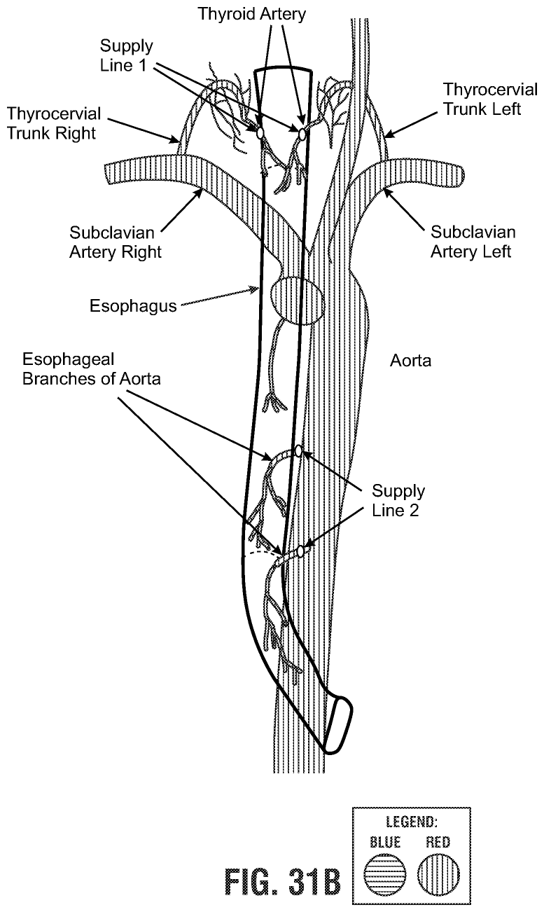

FIG. 31 is another schematic view of the esophagus with labels for different sections thereof and an overview of nearby blood vessels.

FIG. 31A is a schematic view of the primary veins in the esophagus along with locations for positioning collection catheter balloons to isolate and treat diseases therein. RETURN LINE 1 represents MIS for catheter access to the hepatic portal vein. RETURN LINE 2 performs occlusion and retrieval of treated blood and payload via esophageal vein. Access at hepatic portal vein will allow payload draining from the esophageal vein from the bifurcation at the left gastric vein.

FIG. 31B is a schematic view of the primary arteries in the esophagus along with locations for positioning supply catheter balloons to isolate and treat diseases therein. SUPPLY LINE 1 performs occlusion and introduction of treated blood and payload via both esophageal branches of inferior thyroid artery via access from right and left thyrocervial trunk. A couple notes here is that the right and left thyrocervial thyroid arteries are accessed via right and left subclavian arteries, and that depending on location of treatment SUPPLY LINE I could be omitted from treatment. SUPPLY LINE 2 performs occlusion and introduction of treated blood and payload via esophageal branches of aorta via access from the abdominal aorta. Depending on bifurcation of esophageal branches from the aorta one or multi catheters may be used for SUPPLY LINE 2.

### Isolation and Treatment of the Thyroid

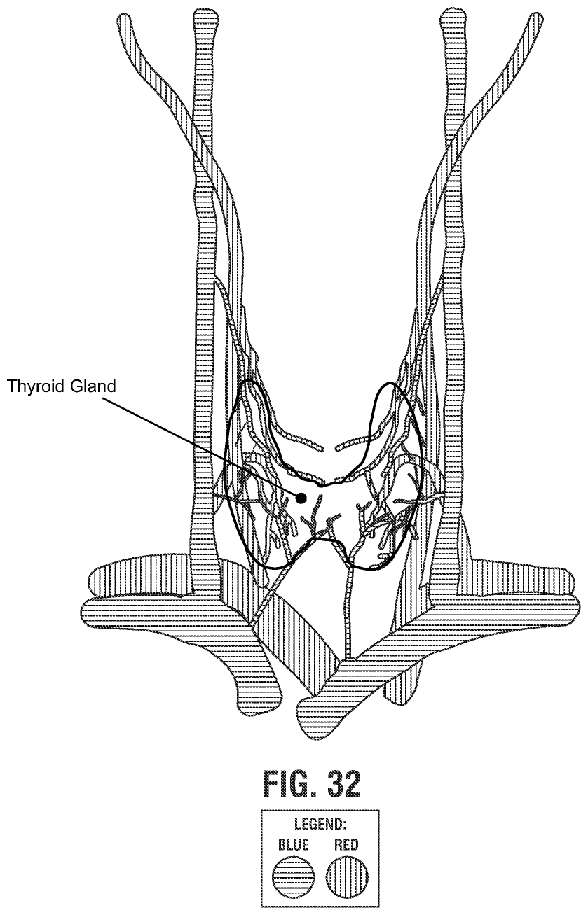

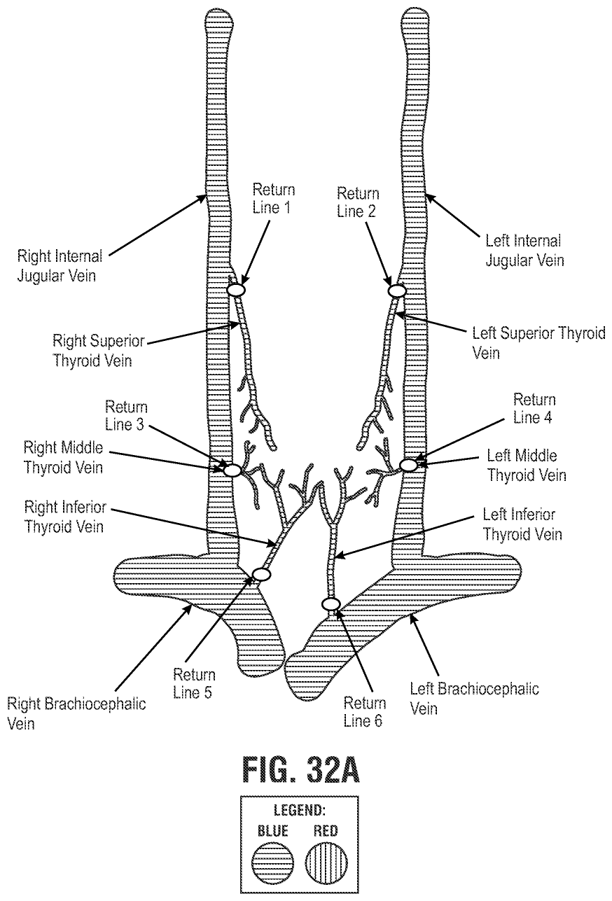

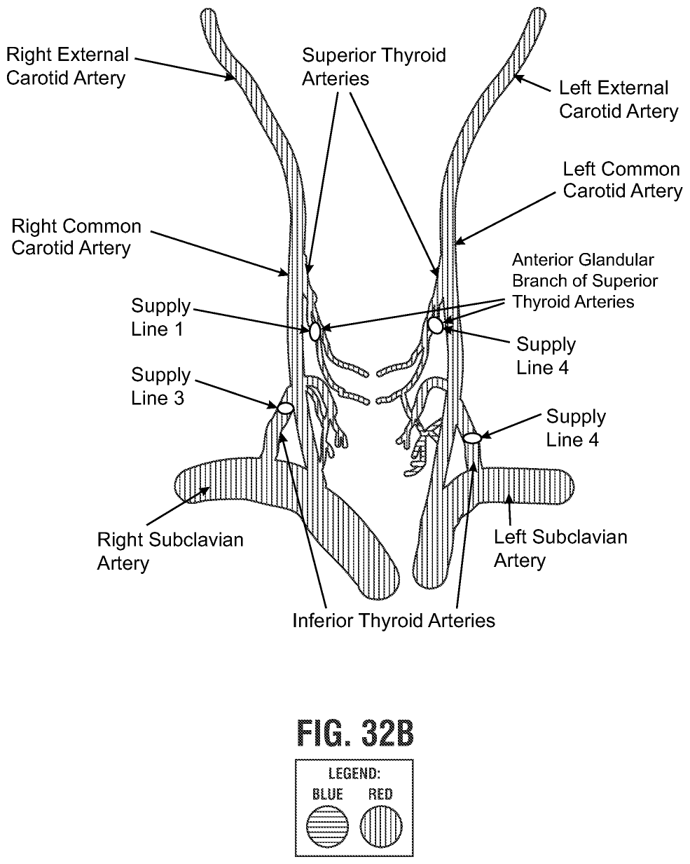

FIG. 32 is another schematic view of the thyroid gland and an overview of nearby blood vessels.

FIG. 32A is a schematic view of the primary veins near the thyroid gland along with locations for positioning collection catheter balloons to isolate and treat diseases therein. RETURN LINES 1 and 2 perform occlusion and retrieval of treated blood and payload via both right and left superior thyroid vein. Access is from the bifurcation of the right and left internal jugular veins. RETURN LINES 3 and 4 provide occlusion and retrieval of treated blood and payload via both right and left middle thyroid vein, and access is from the bifurcation of the right and left internal jugular veins. Finally, RETURN LINES 5 and 6 represent occlusion and retrieval of treated blood and payload via both right and left inferior thyroid vein. Access from bifurcation of the right and left brachiocephalic veins.

FIG. 32B is a schematic view of the primary arteries near the thyroid gland along with locations for positioning supply catheter balloons to isolate and treat diseases therein. SUPPLY LINES 1 and 2, respectively, provide occlusion and introduction of treated blood and payload via both anterior glandular branches of right and left superior thyroid arteries via access from right and left superior thyroid arteries extending from the right and left common carotid arteries. SUPPLY LINES 2 and 3, respectively, perform occlusion and introduction of treated blood and payload via both right and left inferior thyroid arteries via access from right and left subclavian arteries extending from the right and left thyrocervial trunk.

### Exemplary Supply and Return Catheters

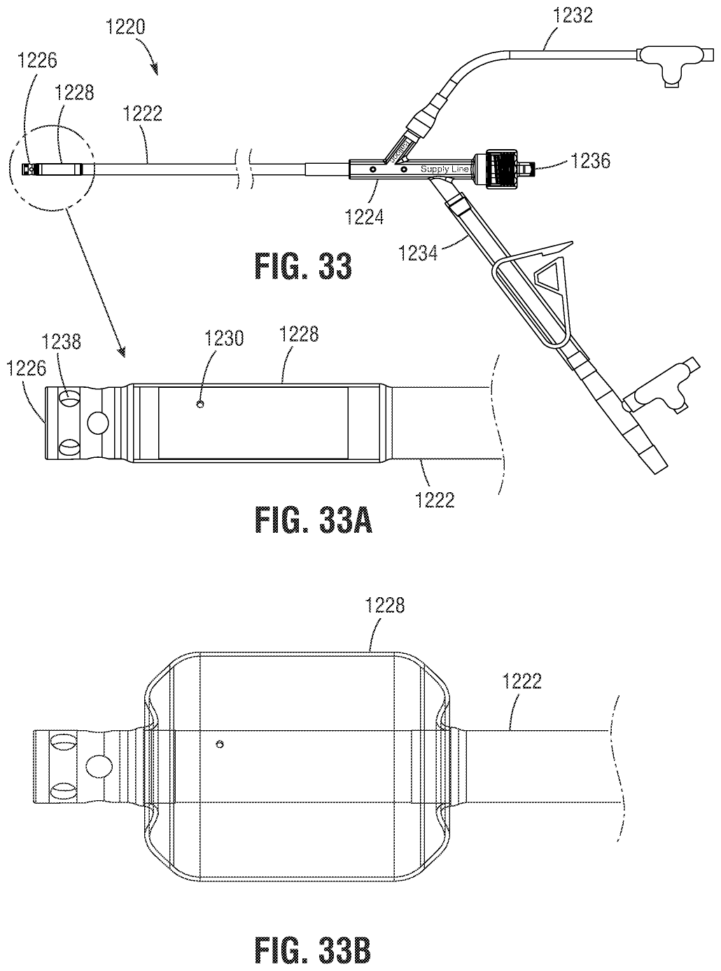

FIG. 33 is a broken elevational view of an exemplary collection catheter 1220 of the present application. The catheter 1220 includes an elongated flexible catheter tube 1222 extending from a proximal handle 1224 and terminating at a distal tip 1226. A balloon 1228 supplied through an internal inflation aperture 1230 is provided closely adjacent to the distal tip 1226. FIG. 33A is an enlargement of the distal balloon 1228, and FIG. 33B shows the balloon in an expanded state.

The proximal handle 1224 includes a number of branched connectors for catheter function. A fluid line 1232 connects to an inner lumen of the catheter tube 1222, and may be used to inflate the balloon 1228. A second fluid line 1234 connects to a different inner lumen of the catheter tube 1222, and may be used to withdraw fluid from the distal tip 1226, and more particularly through flow apertures 1238. Alternatively, a proximal hub 1236 having a luer fitting may be used to withdraw fluid.

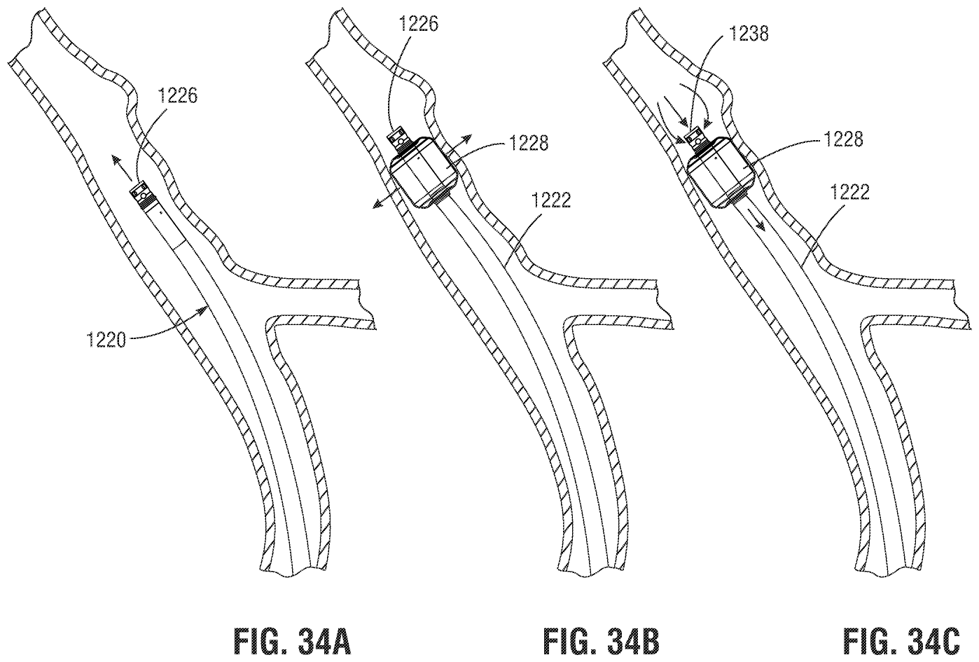

FIGS. 34A-34C illustrate a sequence of deployment of the distal balloon 1228 of the collection catheter 1220 of FIG. 33 from a venous vessel. First, the supply catheter 1220 is introduced into the vasculature and the distal tip 1226 advanced to the target vein adjacent to the target organ. Next, the balloon 1228 is inflated, which isolates the part of the vessel surrounding the distal tip 1226 from the vessel that surrounds the catheter tube 1222. Finally, blood is collected through the flow apertures 1238 as shown, and pulled through an inner lumen of the catheter tube 1222 such as through the action of an external peristaltic or centrifugal pump.

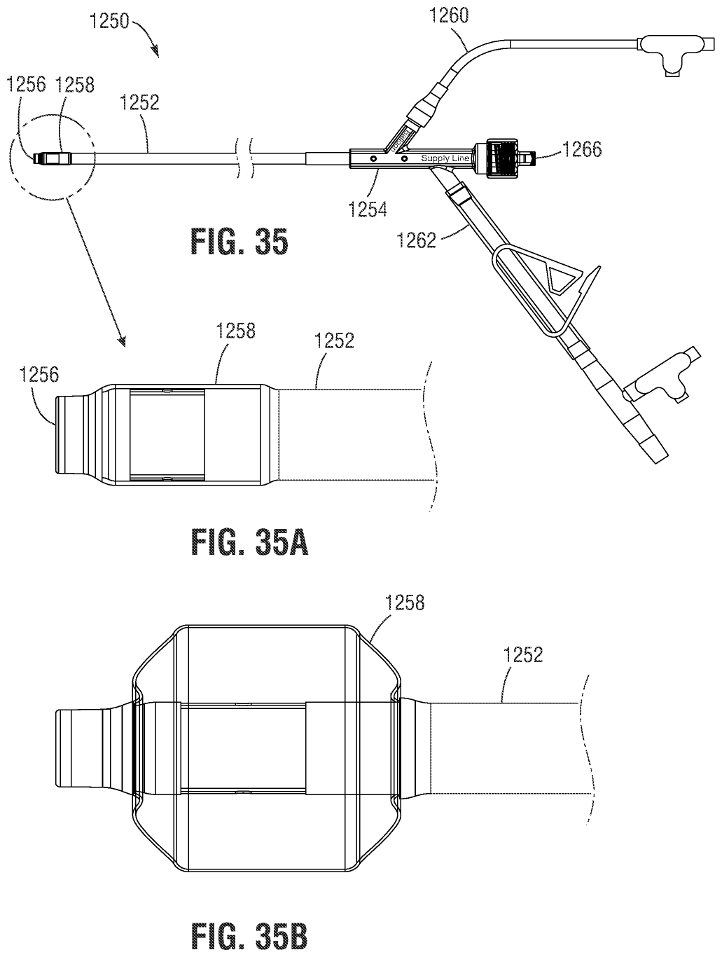

FIG. 35 is a broken elevational view of an exemplary supply catheter 1250 of the present application. The catheter 1250 includes an elongated flexible catheter tube 1252 extending from a proximal handle 1254 and terminating at a distal tip 1256. A balloon 1258 supplied through an internal inflation aperture (not shown) is provided closely adjacent to the distal tip 1256. FIG. 35A is an enlargement of the distal balloon 1258, and FIG. 35B shows the balloon in an expanded state.

The proximal handle 1254 includes a number of branched connectors for catheter function. A fluid line 1262 connects to an inner lumen of the catheter tube 1252, and may be used to inflate the balloon 1258. A second fluid line 1264 connects to a different inner lumen of the catheter tube 1252, and may be used to supply fluid to the distal tip 1256, beyond the balloon 1258. Alternatively, a proximal hub 1266 having a luer fitting may be used to supply fluid.

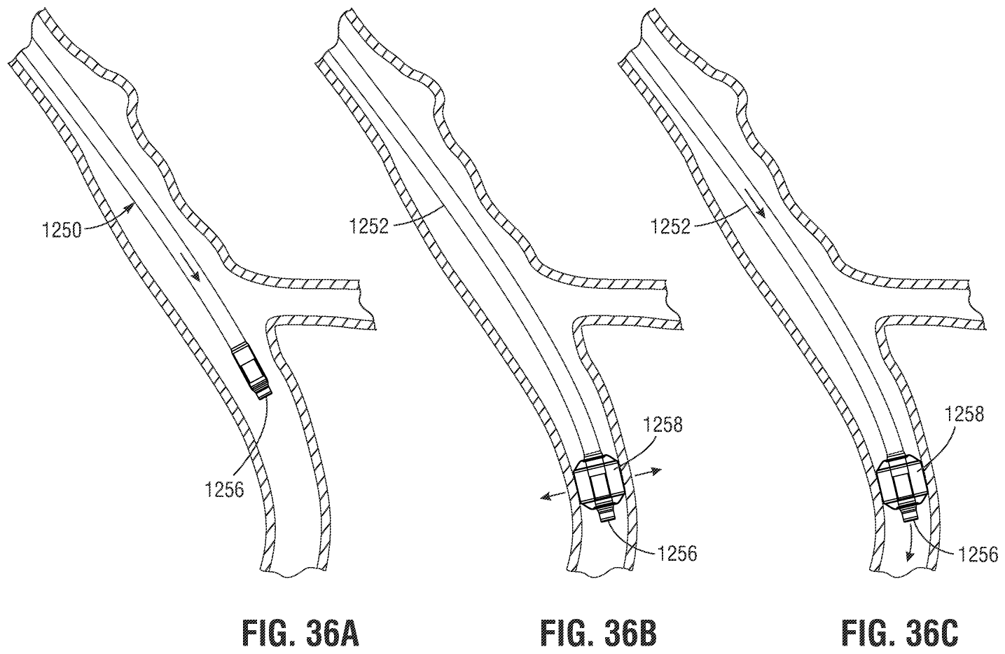

FIGS. 36A-36C illustrate a sequence of deployment of the distal balloon 1258 of the supply catheter 1250 of FIG. 35 and perfusion of fluid distal to the catheter. First, the supply catheter 1250 is introduced into the vasculature and the distal tip 1256 advanced to the target artery. Next, the balloon 1258 is inflated, which isolates the part of the vessel surrounding the distal tip 1256 from the vessel that surrounds the catheter tube 1252. Finally, oxygenated blood and treatment solution are perfused through the catheter tube 1252 and distal tip 1256 as shown, such as through the action of an external peristaltic or centrifugal pump, and delivered beyond the catheter balloon 1258 and to the target organ.

While the invention has been described in its preferred embodiments, it is to be understood that the words that have been used are words of description and not of limitation. Therefore, changes may be made within the appended claims without departing from the true scope of the invention.

What is claimed is:

1. A method for perfusing a treatment solution to part of or all of an organ or tumor within the organ of a human body, comprising:
introducing at least one balloon collection catheter from outside the body into a patient's vasculature and advancing a distal end of the collection catheter to a return location in a venous vessel leading from the organ or tumor, the return location being positioned so as to collect substantially all returned blood from the organ or area of the tumor, a distal tip of the collection catheter having apertures for receiving venous blood therethrough from the venous vessel into a lumen of the collection catheter;
inflating a balloon on the distal end of the collection catheter within the venous vessel, the balloon being sized to occlude the venous vessel;
introducing at least one balloon supply catheter from outside the body into the patient's vasculature and advancing a distal end of the supply catheter to a supply location in an arterial vessel leading to the organ or tumor, the supply location being positioned so as to supply substantially all delivered blood to the organ or area of the tumor, a distal tip of the supply catheter having an aperture for perfusing fluid from a lumen of the supply catheter therethrough into the arterial vessel;
inflating a balloon on the distal end of the supply catheter within the arterial vessel, the balloon being sized to occlude the arterial vessel;
fluidly connecting a proximal end of the collection catheter to a pump;
fluidly connecting a source of treatment solution to the pump; and
fluidly connecting an output conduit from the pump to the lumen of the supply catheter, wherein an isolated circuit is created from the organ or tumor through the collection catheter, from there through the pump, and then through the supply catheter back to the organ or tumor, whereby blood can be removed from just the organ or tumor and returned just to the organ or tumor perfused with the treatment solution.

2. The method of claim 1, wherein the treatment solution is a cancer treatment.

3. The method of claim 1, wherein the organ is a lung, and the treatment solution is selected from the group consisting of an alkylating agent, a taxane, an albumin-bound paclitaxel, an antimetabolite, a vinca alkaloid, and a topoisomerase II inhibitor.

4. The method of claim 1, wherein the organ is the brain, and the treatment solution is a cholinesterase inhibitor for treating Alzheimer's symptoms, and the treatment includes connecting an extracorporeal oxygenator between the proximal end of the collection catheter and the pump and oxygenating the blood prior to returning the blood and treatment solution through the supply catheter back to the organ or tumor.

5. The method of claim 1, wherein the organ is selected from the group consisting of the liver, a kidney, the pancreas, the spleen, the brain, the eyes, the breasts, the brain, the colon, the stomach, the esophagus and the thyroid gland, and the treatment includes connecting an extracorporeal oxygenator between the proximal end of the collection catheter and the pump and oxygenating the blood prior to returning the blood and treatment solution through the supply catheter back to the organ or tumor.

6. The method of claim 1, wherein the organ is selected from the group consisting of the female uterus, the female bladder, the female ovaries, the male bladder, the male prostate, and the male testicles, and the treatment includes connecting an extracorporeal oxygenator between the proximal end of the collection catheter and the pump and oxygenating the blood prior to returning the blood and treatment solution through the supply catheter back to the organ or tumor.

7. The method of claim 1, wherein the organ is selected from the group consisting of the liver, a kidney, the pancreas, the spleen, the colon, the stomach, and the esophagus, and the step of introducing at least one collection catheter and/or the step of introducing at least one supply catheter involves minimally-invasive surgery to install one or more balloon catheters in the Hepatic Portal Vein, and the treatment includes connecting an extracorporeal oxygenator between the proximal end of the collection catheter and the pump and oxygenating the blood prior to returning the blood and treatment solution through the supply catheter back to the organ or tumor.

8. The method of claim 1, wherein there are multiple balloon collection catheters deployed in different vessels.

9. The method of claim 1, wherein there are multiple balloon supply catheters deployed in different vessels.

10. The method of claim 1, wherein the organ is a lung, and the return location and supply location are selected so as to isolate one or more lobes of one lung.

11. The method of claim 1, wherein the organ is a lung, and the return location and supply location are selected so as to isolate one or more segments of one lung.

12. The method of claim 1, wherein the step of introducing at least one balloon collection catheter includes access via a femoral vein.

13. The method of claim 1, wherein the step of introducing at least one balloon supply catheter includes access via a femoral artery.

14. The method of claim 1, wherein the step of introducing at least one balloon supply catheter includes access via the Inferior Vena Cava and through at least one chamber of the heart.

15. The method of claim 1, wherein the balloon collection catheter comprises a fluid line connected to an inner lumen of a catheter tube for inflating the balloon, and a second fluid line connected to a different inner lumen of the catheter tube for withdrawing fluid from the distal tip through multiple flow apertures.

16. A method for perfusing a treatment solution to part of or all of an organ or tumor within the organ of a human body, comprising:
introducing at least one balloon collection catheter from outside the body into a patient's vasculature and advancing a distal end of the collection catheter to a return location in a venous vessel leading from the organ or tumor, the return location being positioned so as to collect substantially all returned blood from the organ or area of the tumor, a distal tip of the collection catheter having apertures for receiving venous blood therethrough from the venous vessel into a lumen of the collection catheter;
inflating a balloon on the distal end of the collection catheter within the venous vessel, the balloon being sized to occlude the venous vessel;
introducing at least one supply catheter from outside the body into the patient's vasculature and advancing a distal end of the supply catheter to a supply location in an arterial vessel leading to the organ or tumor, the supply location being positioned so as to supply substantially all delivered blood to the organ or area of the tumor, a distal tip of the supply catheter having an aperture for perfusing fluid from a lumen of the supply catheter therethrough into the arterial vessel;
fluidly connecting a proximal end of the collection catheter to a pump;
fluidly connecting a source of treatment solution to the pump; and
fluidly connecting an output conduit from the peristaltic pump to the lumen of the supply catheter, wherein an isolated circuit is created from the organ or tumor through the collection catheter, from there through the pump, and then through the supply catheter back to the organ or tumor, whereby blood can be removed from just the organ or tumor and returned just to the organ or tumor and perfused with the treatment solution.

17. The method of claim 16, wherein the organ is the brain, and the treatment solution is a cholinesterase inhibitor for treating Alzheimer's symptoms, and the treatment includes connecting an extracorporeal oxygenator between the proximal end of the collection catheter and the pump and oxygenating the blood prior to returning the blood and treatment solution through the supply catheter back to the organ or tumor.

18. The method of claim 16, wherein the organ is the brain and the organ or tumor within the organ is a brain tumor, wherein the arterial vessel is a capillary and the supply catheter is sized to fit within and occlude the capillary, and the treatment includes connecting an extracorporeal oxygenator between the proximal end of the collection catheter and the pump and oxygenating the blood prior to returning the blood and treatment solution through the supply catheter back to the tumor.

19. The method of claim 16, wherein the organ is the brain and the organ or tumor within the organ is a brain tumor, wherein the treatment includes a controlled supply of oncology drug through at low pressure and flow at the site of the tumor, and the treatment includes connecting an extracorporeal oxygenator between the proximal end of the collection catheter and the pump and oxygenating the blood prior to returning the blood and treatment solution through the supply catheter back to the tumor.

20. The method of claim 16, wherein there are multiple balloon collection catheters deployed in different vessels.
